(19) United States
(12) Patent Application Publication
(10) Pub. No.: US 2025/0009902 A1
(43) Pub. Date: Jan. 9, 2025

(54) ARTIFICIAL ENZYME-BACTERIA SYSTEM AND USES THEREOF

(71) Applicants: NATIONAL UNIVERSITY OF SINGAPORE, Singapore (SG); ZHEJIANG UNIVERSITY, Hangzhou (CN)

(72) Inventors: Xiaoyuan CHEN, Singapore (SG); Zhengwei MAO, Hangzhou, Zhejiang (CN); Fangfang CAO, Hangzhou, Zhejiang (CN)

(21) Appl. No.: 18/688,658

(22) PCT Filed: Sep. 2, 2022
(86) PCT No.: PCT/SG22/50638

§ 371 Date: Mar. 1, 2024

(30) Foreign Application Priority Data
Sep. 2, 2021 (WO) PCT/CN2021/116214

(51) Int. Cl. A61K 47/69; A61K 47/62; A61P 1/00

(52) U.S. Cl. CPC A61K 47/6901; A61K 47/62; A61P 1/00

(57) ABSTRACT

Disclosed is a system comprising an artificial enzyme, a probiotic bacteria and a linker to conjugate the artificial enzyme and probiotic bacteria. Further disclosed are compositions comprising the said system and the uses thereof.

### CROSS-REFERENCE TO RELATED APPLICATIONS

This application claims the benefit of priority of PCT patent application No. PCT/CN2021/116214, filed 2 Sep. 2021, the contents of it being hereby incorporated by reference in its entirety for all purposes.

### FIELD OF THE INVENTION

The present invention generally relates to the field of biotechnology. In particular it refers to artificial enzyme systems and, more particularly, to compositions and methods for treating a gastrointestinal tract disorder using a system comprising an artificial enzyme, a probiotic bacteria and a linker.

### BACKGROUND OF THE INVENTION

Gastrointestinal tract disorders include a myriad of conditions that range from a minor stomach upset or dysbiosis, to a more serious condition such as inflammatory bowel disease (IBD). Inflammatory bowel disease (IBD) describes a family of idiopathic intestinal disorders, such as Crohn's disease (CD) and ulcerative colitis (UC), that causes inflammation of the digestive tract. Such conditions can impose health and economic burdens on communities worldwide, and substantially reduce patients' quality of life.

Present clinical treatments for inflammatory bowel disease (IBD) mainly concentrate on managing the inflammatory symptoms, and generally neglect the underlying causes. Furthermore, these anti-inflammatory and immunosuppressive drugs are not completely effective, and their long-term use may lead to adverse side effects and/or serious complications.

In view of the above, there is a need to provide new and more effective treatments for inflammatory bowel disease (IBD).

### SUMMARY

In one aspect, the present disclosure refers to a system comprising:
a) an artificial enzyme that facilitates a redox reaction;
b) a probiotic bacteria; and
c) a linker to conjugate the artificial enzyme and probiotic bacteria.

In another aspect, the present disclosure refers to a pharmaceutical composition comprising the system as disclosed herein.

In another aspect, the present disclosure refers to a method of treating a disease in a subject in need thereof by administering a therapeutically effective amount of the system or the pharmaceutical composition as disclosed herein.

In another aspect, the present disclosure refers to a method of manufacturing the system as disclosed herein, comprising incubating (i) an artificial enzyme conjugated to a linker; and (ii) a probiotic bacteria, wherein the linker conjugates the artificial enzyme and probiotic bacteria.

### BRIEF DESCRIPTION OF THE DRAWINGS

The invention will be better understood with reference to the detailed description when considered in conjunction with the non-limiting examples and the accompanying drawings, in which:

FIG. 1 shows the schematics of the artificial enzyme-probiotic bacteria system. (a) illustrates the general structures of the artificial enzyme-probiotic bacteria system as disclosed herein. (b) An exemplary artificial enzyme-probiotic bacteria system, wherein the linker has a phenylboronic acid functional group. (c) Another exemplary artificial enzyme-probiotic bacteria system, wherein the linker has a phenylboronic acid functional group, and a PEG. FIG. 1 provides exemplary structures of the artificial enzyme-probiotic system as disclosed herein.

FIG. 2 is an image showing exemplary preparation of the artificial enzyme-probiotic bacteria system and their microenvironment regulations in inflammatory bowel disease (IBD) treatment. (a) BL@B-SA is composed of artificial enzymes (for example, Fe SA), probiotic bacteria (for example, Bifidobacterium longum (BL)), and linker (for example, C18-PEG-B) to conjugate the artificial enzyme and probiotic bacteria. In this example, Fe SA is fabricated through the pyrolysis of ZIF-8 encapsulated Fe precursors (Fe@MOF). The linker C18-PEG-B comprises a hydrophobic C18 group that can be attached to Fe SA surface through noncovalent interactions and a phenylboronic acid functional group to capture bacteria via boronic acid vicinal-diol-based click reaction. (b) BL@B-SA can mimic SOD and CAT antioxidant enzymes as well as function as antioxidant molecules to scavenge multiple reactive oxygen species (ROS) to regulate the fate of cells and microbes. (c) Clinically used therapeutics for IBD encounter low efficacy and severe systemic toxicity, due to the irreversible depletion and inevitable off-target effects of clinical drugs as well as the inherent vulnerability of probiotics to oxidative stress. In contrast, the artificial enzymes-probiotic bacteria system can treat gastrointestinal tract disorders such as inflammatory bowel disease (IBD) by using the combination of targeted antioxidant therapy of SAzymes and the fast microbiome regulation of durable probiotic bacteria. FIG. 2 illustrates the preparation of artificial enzyme-probiotic bacteria system and how they interact with the gastrointestinal microenvironment.

FIG. 3 has Transmission Electron Microscopy (TEM) images and graphs that represent the features of the artificial enzyme-probiotic bacteria system. (a) Transmission Electron Microscopy (TEM) images, (b) aberration-corrected high-angle annular dark-field scanning transmission electron microscopy (HAADF-STEM) images to show a portion of the artificial enzyme-probiotic bacteria system, wherein (c) is an enlarged image of the iron single atom (Fe SA). Single iron (Fe) atoms are identified by white circles. (d) A HAADF-STEM image and corresponding element maps showing the distribution of Fe (bottom right), C (top right), and N (bottom left). (e) A XANES spectra and (f) Fourier transform (FT) of the Fe K-edge of the Fe K-edge of Fe foil, Fe₂O₃, FePc and Fe SA. (g) The corresponding EXAFS fitting curves of Fe SA at R space. (h) An EXAFS fitting curves of Fe SA at k space. TEM images of (i, j) B-SA and (k, l) BL@B-SA under different magnification. FIG. 3 shows the characterization and reactive oxygen species (ROS)-scavenging ability of the artificial enzyme-probiotic bacteria system

FIG. 4 shows results of the multiple ROS-scavenging abilities in vitro. Graphs of (a) O₂″, (b) H₂O₂ and (c) ·OH-scavenging ability of B-SA. Graphs showing comparison of the (d) O₂″, (e) H₂O₂ and (f) ·OH-scavenging ability of different materials. ns: no significance, *P<0.05, **P<0.01 and ***P<0.001 versus the BL group. (g) A column graph showing the relative viability of BL under the protection of different concentrations of B-SA in 200 μM H₂O₂ for 2 h. ns, no significance, *P<0.05, **P<0.01 and ***P<0.001 versus the BL group. Graphs showing the relative viability of BL under the protection of BL@B-SA₅₀ in the (h) absence or (i) presence of 200 μM H₂O₂ for different periods of time. ns, no significance, *P<0.05, **P<0.01 and ***P<0.001 versus 0 h. (j) A column graph showing the relative cell viability of HT29 cells with various treatments. *P<0.05, **P<0.01 and ***P<0.001 versus the untreated group. (k) A column graph showing the relative viability of HT29 cells with various treatments in the presence of 200 μM H₂O₂. ns, no significance, *P<0.05, **P<0.01 and ***P<0.001 versus the control group. ###P<0.001. (l) A column graph showing the flow cytometry measurement of intracellular ROS in HT29 cells after treatment with different materials followed by 400 μM H₂O₂. Intracellular ROS was indicated by DCFH-DA. ns, no significance, *P<0.05, **P<0.01 and ***P<0.001 versus the untreated group. ###P<0.001. (m) Confocal fluorescence images of ROS levels in HT-29 cells with different treatments. The cells were stained by ROS probe dichlorofluorescein diacetate (DCFH-DA) and Hoechst. FIG. 4 shows that exemplary artificial enzyme-probiotic linker system promotes the growth of HT29 cells and protects them from ROS attack.

FIG. 5 has graphs and images to show the stability and accumulation of BL@B-SA in inflamed intestine. Column graphs showing ROS-scavenging abilities of BL@B-SA₅₀ and BL in the simulated gastrointestinal environments of (a) simulated gastric fluid (SGF), (b) simulated intestinal fluid (SIF) and (c) simulated inflammatory colon fluid (SICF). Column graphs showing corresponding viability of BL in the (d) SGF, (e) SIF and (f) SICF. ns, no significance, *P<0.05, **P<0.01 and ***P<0.001. (g) Representative photoacoustic (PA) images and intensity of PA signals of the intestinal tracts after oral gavage of BL@B-SA and B-SA for 6 h. ns, no significance, *P<0.05, **P<0.01 and **P<0.001. FIG. 5 shows that the modification in the exemplary artificial enzyme-probiotic bacteria system improves ROS-scavenging and inflammation-reduction.

FIG. 6 shows results of the in vivo toxicology testing of BL@B-SA. (a) Hematological and (b) blood biochemical analysis of the healthy mice orally administered with or without BL@B-SA₅₀ for 28 days. 1 refers to control mice. 2 refers to mice treated with BL@B-SA₅₀. (c) Body weights of the healthy mice orally administered with or without BL@B-SA₅₀ during 28 days. Data is presented as mean±s.d. (n=5/group). (d) Representative photographs and (e) Hematoxylin and eosin (H&E) images obtained from the major organs of the control mice and healthy mice with different treatments at 28 days. Scale bar: 50 μm.

FIG. 7 shows images and graphs of the efficacy of BL@B-SA against ulcerative colitis (UC). (a) A schematic of the experimental setup. C57BL/6 mice are provided with water or 3% DSS-containing water for 4 days. On days 4, 5, 6 and 7, mice were orally administered with Medium, B-SA (1.25 mg/kg), BL (2.5×10⁸ CFU/kg), BL (2.5×10⁸ CFU/kg)+B-SA (1.25 mg/kg), or BL@B-SA₅₀ (BL: 2.5×10⁸ CFU/kg, B-SA: 1.25 mg/kg). (b) Graph showing the daily bodyweight changes in each group for 9 days. Data were normalized as a percentage of the body weight at day 0. ns, no significance, *P<0.05, **P<0.01 and ***P<0.001 versus the control group. (c) Graph showing the changes in Disease Activity Index (DAI) for 9 days, which is the summation of the stool consistency index (0-3), fecal bleeding index (0-3), and weight loss index (0-4). ns, no significance, *P<0.05, **P<0.01 and ***P<0.001 versus the control group. (d) Column graph showing the colon lengths of mice with indicated treatments on day 8. ns, no significance, *P<0.05, **P<0.01 and ***P<0.001 versus the control group. (e) Photos of H&E staining and immunohistochemical analysis of colonic sections of mice on day 8 after indicated treatments. Column graphs showing (f) relative ROS and (g) typical inflammatory cytokines of colons of mice on day 8 after indicated treatments. ns, no significance, *P<0.05, **P<0.01 and ***P<0.001 versus the control group. ^{#}P<0.05. (h) Graph showing survival rate of mice in two weeks after the indicated treatments. Data were presented as mean±s.d. (n=5/group). (i) Column graph showing a-diversity of the fecal microbiome illustrated by Shannon's index. ns, no significance, *P<0.05, **P<0.01 and ***P<0.001 versus the BL@B-SA₅₀ group. (j) An image of a NMDS plot showing β-diversity of fecal microbiome. (k) Column graphs showing relative abundance of select taxa. For Verrucomicrobiota and Firmicutes, ns, no significance, *P<0.05, **P<0.01 and ***P<0.001 versus the control group. ns, no significance and #P<0.05. For Firmicutes/Bacteroidota, ns, no significance and ^{#}P<0.05. For Lachnospiraceae, **P<0.05 and **P<0.001 versus the BL@B-SA₅₀ group. (1) Images of colons (top) and rectal areas (bottom) of mice on day 8 after indicated treatments. FIG. 7 shows that the exemplary artificial enzyme-probiotic bacteria system ameliorates DSS-induced ulcerative colitis.

FIG. 8 shows images and graphs of comparing ulcerative colitis (UC) therapy of BL@B-SA with different proportions of BL and B-SA. (a) A schematic of the experimental setup. C57BL/6 mice were provided with water or 3% DSS-containing water for 4 days. On days 4, 5, 6 and 7, mice were orally administered with medium, BL@B-SA₂₅ (BL: 2.5×10⁸ CFU/kg, B-SA: 0.625 mg/kg), BL@B-SA₅₀ (BL: 2.5×10⁸ CFU/kg, B-SA: 1.25 mg/kg), or BL@B-SA₁₀₀ (BL: 2.5×10⁸ CFU/kg. B-SA: 2.5 mg/kg). (b) Graph showing the daily body weight changes in each group for 9 days. Data were normalized as a percentage of the body weight at day 0. *P<0.05, **P<0.01 and ***P<0.001 versus the control group. (c) Graph showing the changes in Disease Activity Index (DAI), which is the summation of the stool consistency index (0-3), fecal bleeding index (0-3), and weight loss index (0-4), ***P<0.001 versus the control group. (d) Column graph showing colon lengths of mice with indicated treatments on day 8, ***P<0.001 versus the colitis group. ns, no significance and ^{#}P<0.05 versus the health group. (e) Images of colons of mice with indicated treatments on day 8, respectively. Data were presented as mean±s.d. (n=5/group). FIG. 8 shows that BL@B-SA₅₀ and BL@B-SA₁₀₀ conferred better protection for the animals.

FIG. 9 shows images and graphs comparing UC therapy of BL@B-SA₅₀ with different dosages. (a) A schematic of the experimental setup. C57BL/6 mice were provided with water or 3% DSS-containing water for 4 days. On days 4, 5, 6 and 7, mice were orally administered with 0, 0.625 mg/kg (BL: 1.25×10⁸ CFU/kg, B-SA: 0.625 mg/kg), 1.25 mg/kg (BL: 2.5×10⁸ CFU/kg, B-SA: 1.25 mg/kg), 2.5 mg/kg (BL: 5×10⁸ CFU/kg, B-SA: 2.5 mg/kg) and 5 mg/kg BL@B-SA (BL: 10×10⁸ CFU/kg, B-SA: 5 mg/kg) BL@B-SA₅₀. (b) Graph showing daily body weight changes in each group for 9 days. Data were normalized as a percentage of the body weight at day 0. *P<0.05 and ***P<0.001 versus the control group that without giving BL@B-SA₅₀. (c) Graph showing changes in Disease Activity Index (DAI), which is the summation of the stool consistency index (0-3), fecal bleeding index (0-3), and weight loss index (0-4). ns, no significance, **P<0.01 and ***P<0.001 versus the control group. (d) Column graph showing colon lengths of mice with indicated treatments on day 8. *P<0.05, **P<0.01 and ***P<0.001 versus the control group. (e) Images of colons of mice with indicated treatments on day 8, respectively. Data were presented as mean±s.d. (n=5/group). FIG. 9 shows the different doses of the exemplary artificial enzyme-probiotic system that can have minimal side effects.

FIG. 10 shows images and graphs comparing ulcerative colitis (UC) therapy between BL@B-SA and clinical drugs. (a) A schematic of the experimental setup. C57BL/6 mice were provided with water or 3% DSS-containing water for 4 days. On days 4, 5, 6 and 7, mice were orally administered with Medium, 5-ASA (30 mg/kg), DEX (1 mg/kg), MPS (1 mg/kg) or BL@B-SA₅₀ (BL: 2.5× 10⁸ CFU/kg, B-SA: 1.25 mg/kg). (b) Graph showing daily body weight changes in each group for 9 days. Data were normalized as a percentage of the body weight at day 0. ns, no significance, *P<0.05, **P<0.01 and ***P<0.001 versus the control group. (c) Graph showing changes in Disease Activity Index (DAI), which is the summation of the stool consistency index (0-3), fecal bleeding index (0-3), and weight loss index (0-4). ns, no significance, *P<0.05, **P<0.01 and ***P<0.001 versus the control group. (d) Column graph showing colon lengths of mice with indicated treatments on day 8. ns, no significance, *P<0.05, **P<0.01 and ***P<0.001 versus the control group. (e) Images of colons of mice with indicated treatments on day 8, respectively. Data were presented as mean±s.d. (n=5/group). FIG. 10 shows that the exemplary artificial enzyme-probiotic system exhibited enhanced efficacy against DSS-induced colitis in comparison to other conventional IBD therapeutics such as 5-ASA, MPS and DEX.

FIG. 11 shows images and graphs characterizing the effects of Crohn's disease (CD) therapy with BL@B-SA. (a) A schematic of the experimental setup. C57BL/6 mice were pre-sensitized by absorption of TNBS solution through the skin. After one week, TNBS solutions were slowly administered into the colon lumen of mice to induce CD. Then, mice were orally administered with Medium or B-SA (1.25 mg/kg), BL (2.5×10⁸ CFU/kg), BL (2.5×10⁸ CFU kg-1)+B-SA (1.25 mg/kg), BL@B-SA₅₀ (BL: 2.5×10⁸ CFU/kg, B-SA: 1.25 mg/kg) 1.25 mg/kg (BL: 2.5×10⁸ CFU/kg. B-SA: 1.25 mg/kg) for four days. (b) Graph showing daily bodyweight development after administering TNBS solution into the colon lumen. Data were normalized as a percentage of the bodyweight at day 8. ns, no significance, *P<0.05, **P<0.01 and ***P<0.001 versus the control group. (c) Graph showing changes in Disease Activity Index (DAI), which is the summation of the stool consistency index (0-3), fecal bleeding index (0-3), and weight loss index (0-4). ns, no significance, *P<0.05, **P<0.01 and ***P<0.001 versus the control group. (d) Column graph showing colon lengths of mice with indicated treatments on day 13. ns, no significance, *P<0.05, **P<0.01 and ***P<0.001 versus the control group. (e) Images of colons of mice with indicated treatments on day 8, respectively. Data were presented as mean±s.d. (n=5/group). FIG. 11 shows that the exemplary artificial enzyme-probiotic system can be used to treat Crohn's disease.

FIG. 12 shows images and graphs of the effects of ulcerative colitis (UC) therapy using BL@B-SA in beagle dogs. (a) A schematic of the experimental setup. Beagle dogs were perfused with 7% 2 mL/kg acetic acid for 120 s. After one day, the UC group with treatment was orally administered with BL@B-SA₅₀ (BL: 1×10⁸ CFU/kg, B-SA: 0.5 mg/kg) for 6 consecutive days. (b) A column graph showing white blood cell (WBC) contents of dogs with indicated treatments on day 7, respectively. ns, no significance, *P<0.05, **P<0.01 and ***P<0.001 versus the control group. (c) Images of endoscopic photographs of dogs with different treatments during 7 days. (d) Images of colons obtained from dogs with indicated treatments on day 7. (e) Photos of H&E-staining and immunohistochemical analysis of colons obtained from dogs with indicated treatments on day 7. (f) Column graphs showing typical inflammatory cytokines of colons obtained from dogs with indicated treatments on day 7. ns, no significance, *P<0.05, **P<0.01 and ***P<0.001 versus the control group. Scale bar: 50 μm. FIG. 12 shows that the exemplary artificial enzyme-probiotic system is effective in treating ulcerative colitis (UC) in dogs.

FIG. 13 are the biosafety results of BL@B-SA on dogs. Representative hematoxylin and eosin (H&E)-stained sections of (a) the major organs (heart, liver, spleen, lung and kidneys) and (b) the major digestive tract (esophagus, stomach, small intestine, jejunum and ileum) of dogs from indicated groups on day 7. Scale bar: 100 μm. FIG. 13 shows that the exemplary artificial enzyme-probiotic bacteria system is safe and suitable for dogs.

### DETAILED DESCRIPTION

Different disorders of the gastrointestinal tract have different etiology. One example of a common gastrointestinal tract disorder is inflammatory bowel disease (IBD), such as that in Crohn's disease (CD) and ulcerative colitis (UC). Emerging evidence suggests that inflammatory bowel disease (IBD) is caused by the dysfunction of intestinal mucosal barrier and disturbances in the bacterial microbiota, resulting in hyperactive immune responses such as elevated expression of free radicals and inflammatory factors.

As used herein, the term “free radical” refers to a molecule containing one or more unpaired electrons in atomic orbit and is capable of independent existence. Free radicals are products of cellular metabolism, and can include, but are not limited to, reactive oxygen species (ROS). Due to the presence of the one or more unpaired electrons, free radicals are highly reactive species and can either donate the one or more unpaired electrons to another molecule or accept one or more unpaired electrons to another molecule. This causes the other molecule to become a free radical and thereby starts a chain reaction, which can cause negative effects in cells when the level of free radicals becomes unbalanced. It is therefore no surprises that free radicals have been implicated in many different conditions and diseases, including gastrointestinal tract disorders and inflammatory disorders.

Current clinical therapies mainly focus on alleviating the disease-related symptoms by suppressing the intestinal inflammatory burden. Despite the progress made in the development of anti-inflammatory drugs, conventional treatments are not highly effective. In addition, long-term use of such drugs may result in altered drug metabolism and greater dependency. Anti-inflammatory drugs can also cause unwanted side effects due to off-target effects, sometimes resulting in serious complications. More critically, most of the anti-inflammatory drugs do not target the underlying causes of inflammatory bowel disease (IBD): the dysfunction of intestinal mucosal barrier and disturbances in the bacterial microbiota. Neglecting treatment of the underlying causes of inflammatory bowel disease (IBD) can further intensify the inflammatory response.

Probiotic bacteria have been shown to inhibit pathogen colonization by accumulating in the intestinal site, thereby positively modulating the balance of bacterial composition. This promotes intestinal mucosal repair, thus actively shaping a healthy immune system. Currently, probiotic bacteria are delivered using physical coatings that can resist attacks from H+, protease and antibiotics in the gastrointestinal tract. However, the efficacy of probiotic bacteria can still be adversely affected by environmental factors such as incompatible pH, temperature or the lack of antioxidant enzymes. For example, a strain of probiotic bacteria can work in acidic conditions in the stomach, but the efficacy is decreased in non-acidic conditions in the intestine. For example, Bifidobacterium longum (BL) is an anaerobic bacterium that has been shown to reduce the severity of colitis. In addition, hyperactive reactive oxygen species (ROS) present in inflamed gastrointestinal tract is also detrimental to the metabolism of anaerobic bacteria.

Artificial enzymes are alternatives to small molecular antioxidants. Artificial enzymes have lower cost, higher stability and durability, and can be modulated to have enzyme-mimicking activities. Single-atom artificial enzymes have ultrahigh catalytic activity and robust stability, and can mimic SOD and CAT to efficiently scavenge multiple ROS of superoxide radical (O₂″) and hydrogen peroxide (H₂O₂) for IBD treatment. Moreover, they can also scavenge hydroxyl radical (·OH) to further improve their outcomes.

Thus, there is a need for new and more effective treatments for gastrointestinal tract disorders such as inflammatory bowel disease (IBD) that can effectively target any inflamed organ in the gastrointestinal tract and reshape the microenvironment by: suppressing inflammation, remodeling intestinal barrier function and modulating gut microbiome in the infected tissues. In addition, there is also a need to address the inherent vulnerability of probiotic bacteria to oxidative stress so that the benefits of probiotic bacteria can be harnessed and used in treating gastrointestinal tract disorders.

In view of the above, the inventors have developed a system comprising:
a) an artificial enzyme that facilitates a redox reaction;
b) a probiotic bacteria; and
c) a linker to conjugate the artificial enzyme and probiotic bacteria.

In one example, the system comprises a) an artificial enzyme that facilitates a redox reaction and b) a probiotic bacteria, wherein the probiotic bacteria and artificial enzyme are conjugated (bound) together via a linker. In another example, the system comprises a linker conjugating a) an artificial enzyme that facilitates a redox reaction and b) a probiotic bacteria.

As used herein, the term “artificial enzyme” refers to a synthetic protein or material that does not occur in nature. An artificial enzyme can possess the catalytic functions of a naturally occurring enzyme. An artificial enzyme can also be a compound that does not have an intrinsic catalytic domain, but can participate in a chemical reaction by reacting with free radicals.

In one example, the artificial enzyme comprises a nanozyme, or a genetically-engineered enzyme or a biorthogonal catalyst. In another example, the artificial enzyme is a nanozyme.

As used herein, the term “nanozyme” refers to a synthetic molecule made of nanomaterials that comprises intrinsic enzyme-like activity. Nanozyme is an artificial enzyme that is made from nanomaterials such as, but not limited to, single-atom catalysts, metal cluster, metal-organic framework, and carbon-based nanomaterials. The nanozyme functions by mimicking the catalytic sites of natural enzymes, wherein the functions can be adjusted accordingly by changing the active metal element and coordination chemical structure. In one example, the nanozyme can be any one of, but not limited to a single-atom nanozyme (SAzyme), a carbon-based nanozyme and a cluster-based nanozyme. In another example, the nanozyme is a single-atom nanozyme (SAzyme).

As it would be appreciated, the term “single-atom nanozyme” or “SAzyme” as used herein refers to a single-atom catalyst with enzyme-like activity. Single-atom catalysts are defined as catalysts in which all active metal species exist as isolated single atoms stabilized by the support of or by alloying with another metal. In one example, the SAzyme can be any one of, but not limited to iron single-atom nanozyme, copper single-atom nanozyme, zinc single-atom nanozyme, cobalt single-atom nanozyme, gold single-atom nanozyme, platinum single-atom nanozyme, nickel single-atom nanozyme, or a combination of any of the aforementioned. In another example, the SAzyme is an iron single-atom nanozyme (SAzyme).

Similar to the nanozyme, the single-atom nanozyme functions can also be adjusted by changing the coordination chemical structure. In one example, the SAzyme comprises a Fe—N pentacoordination structure, a tetracoordination structure, or a S/P doping structure. In another example, the SAzyme comprises a Fe—N pentacoordination structure.

In the present application, the artificial enzyme facilitates a redox reaction. As used herein, the term “redox reaction” refers to a reaction that involves the exchange of one or more electrons in a molecule. A redox reaction is otherwise known as an oxidation-reduction reaction. In one example, a redox reaction is a reduction reaction. Reduction occurs when there is a gain of one or more electrons in a molecule, a loss of one or more oxygen in a molecule, or a gain of one or more hydrogen in a molecule. In another example, a redox reaction is an oxidation reaction. Oxidation occurs when there is a loss of one or more electrons in a molecule, a gain of one or more oxygen in a molecule, or a loss of one or more hydrogen in a molecule. Oxidation reaction can result in the production of free radicals.

In another example, a redox reaction is an antioxidation reaction. As it would be appreciated, an antioxidation reaction is similar, if not identical, to a reduction reaction. Antioxidants are known to enable antioxidation reactions. As used herein, the term “antioxidant” refers to any natural or synthetic compounds that inhibit or reduce oxidation reactions. Antioxidants can remove free radicals so that a cell can maintain a certain balance of free radical levels. The action of the antioxidants helps to protect the cells from the negative effects of free radicals. In one example, an antioxidant is an enzymatic antioxidant such as a reactive oxygen species (ROS) scavenging enzyme. In another example, an antioxidant is an antioxidant molecule, which is a low-molecular-weight compound that comprises reactive oxygen species (ROS) scavenging ability.

Therefore, an artificial enzyme facilitating a redox reaction essentially refers to the artificial enzyme being able to initiate, start, catalyze or enable a redox reaction as described above.

The system described herein comprises an artificial enzyme that comprises reactive oxygen species (ROS) scavenging ability to scavenge one or more reactive oxygen species (ROS). The terms “reactive oxygen species” or “ROS” as used herein refer to highly reactive molecules and free radicals derived from oxygen molecules, and are usually generated from oxidative metabolism in a cell. Examples of reactive oxygen species include, but are not limited to superoxide radical (O₂·—), hydrogen peroxide (H₂O₂) and hydroxyl radical (·OH).

In one example, the system comprises an artificial enzyme that comprises reactive oxygen species (ROS) scavenging ability of a reactive oxygen species (ROS) scavenging enzyme. The reactive oxygen species (ROS) scavenging enzyme can be any ROS scavenging enzyme. In another example, the artificial enzyme mimics a reactive oxygen species (ROS) scavenging enzyme can be any one of, but not limited to superoxide dismutase (SOD), catalase (CAT), and glutathione peroxidase (GPx). In another example, the system comprises an artificial enzyme that comprises reactive oxygen species (ROS) scavenging ability of an antioxidant molecule. The antioxidant molecule can be any antioxidant molecule that is known in the art. In another example, the artificial enzyme comprises reactive oxygen species (ROS) scavenging ability of an antioxidant molecule can be any one of, but not limited to glutathione (GSH), cysteine (Cys), polyphenol and vitamin C.

In addition to the artificial enzyme, the system also comprises a probiotic bacteria. As used herein, the term “probiotic bacteria” refers to any bacteria that beneficially affects a host subject by improving microbial balance. The benefits of probiotic bacteria include, but are not limited to supporting a healthy gut microbiota, supporting a healthy digestive tract and supporting a healthy immune system. The system can comprise any known probiotic bacteria.

In one example, the probiotic bacteria is from a genus from any one of, but not limited to Bifidobacterium, Lactobacillus, Komagatacibacter, and Leuconostoc. In another example, the probiotic bacteria is Bifidobacterium.

It would be appreciated that the Bifidobacterium genus would comprise many different species, wherein the probiotic bacteria would not be limited to a particular species of Bifidobacterium. In one example, the Bifidobacterium can be any one of, but not limited to Bifidobacterium longum, Bifidobacterium bifidum, Bifidobacterium breve, Bifidobacterium adolescentis, Bifidobacterium infantis, and Bifidobacterium animalis. In another example, the Bifidobacterium is Bifidobacterium longum.

The system further comprises a linker to conjugate the artificial enzyme and probiotic bacteria. As used herein, the term “conjugate” refers to the joining together of two or more chemical elements or components, by whatever means including chemical conjugation or recombinant means. In one example, the linker comprises a phenylboronic acid functional group. As used herein, the term “phenylboronic acid functional group” refers to the following chemical structure

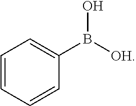

In one example, the phenylboronic acid functional group conjugates to the probiotic bacteria. “B” as shown in the chemical structure

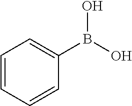

refers to boron.

In addition to the phenylboronic acid functional group, the linker can further comprise, but not limited to, polyethylene glycol (PEG), a single stranded DNA (ssDNA), polysialic acid (PSA), starch, hydroxyalkyl starch (HAS), hydroxylethyl starch (HES), carbohydrate, polysaccharides, pullulane, chitosan, hyaluronic acid, chondroitin sulfate, dermatan sulfate, dextran, carboxymethyl-dextran, polypropylene glycol (PPG), polyoxazoline, polyacryloylmorpholine, polyvinyl alcohol (PVA), polyphosphazene, polyoxazoline, polyethylene-co maleic acid anhydride, polystyrene-co maleic acid anhydride, poly(1-hydroxymethylethylene hydroxymethylformal) (PHF), or 2-methacryloyloxy-2′-ethyltrimethylammoniumphosphate (MPC). Some of these compounds, for example but not limited to polyethylene glycol (PEG), carbohydrate, polysaccharides, chitosan, or dextran, contain active aminooxy group that can react with carboxyl phenylboric acid through amidation reaction, or contain hydroxyl group can react with carboxyl phenylboric acid through esterification reaction. In one example, the linker further comprises polyethylene glycol (PEG) or a single stranded DNA (ssDNA).

In one example, the linker further comprises polyethylene glycol (PEG). Polyethylene glycol (PEG) can be added to the linker by the process of PEGylation. As described herein, the term “PEGylation” refers to the addition of polyethylene glycol to a molecule or material, for example but not limited to a phenylboronic acid functional group, a peptide, a protein or a drug. PEGylation occurs by covalent conjugation of polyethylene glycol (PEG) to the molecules or materials. In one example, the polyethylene glycol (PEG) is linear or branched. In another example, the polyethylene glycol (PEG) is linear. Different forms of polyethylene glycol (PEG) can exist depending on the number of conjugated carbon chain length. The PEG as used herein can be denoted as Cn-PEG, wherein Cn-PEG contains Cn alkyl chain to modify artificial enzymes through noncovalent interactions and PEG (molecular weight (Mw)=2000) linker. “n” represents any number of carbon in the alkyl chain. The number of carbon in the PEG can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more. Therefore, the PEG can be C1-PEG, C2-PEG, C3-PEG, C4-PEG, C5-PEG, C6-PEG, C7-PEG, C8-PEG, C9-PEG, C10-PEG, C11-PEG, C12-PEG, C13-PEG, C14-PEG, C15-PEG, C16-PEG, C17-PEG, C18-PEG, C19-PEG, C20-PEG, C21-PEG, C22-PEG, C23-PEG, C24-PEG, C25-PEG, C26-PEG, C27-PEG, C28-PEG, C29-PEG, or C30-PEG. In another example, the PEG is C12-PEG or C18-PEG. In one example, the linker comprises a phenylboronic acid functional group and a PEG. In a more particular example, the linker comprises a phenylboronic acid functional group and a C12-PEG. In another particular example, the linker comprises a phenylboronic acid functional group and a C18-PEG. In one example, the linker comprises the structure

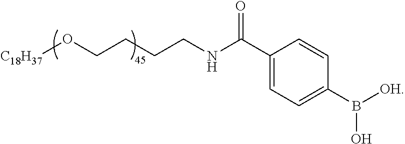

In another example, the linker further comprises a single stranded DNA (ssDNA). The single stranded DNA (ssDNA) can be 10-50 residues in length. The single stranded DNA (ssDNA) can also be, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 residues in length.

In one example, the system described herein is produced by first synthesizing the artificial enzyme and linker, followed by incubating the probiotic bacteria with the artificial enzyme and linker. It would be appreciated that the number of probiotic bacteria can be increased or decreased based on a fixed amount of artificial enzyme and linker, or that the amount of artificial enzyme and linker can be increased or decreased based on a fixed number of probiotic bacteria.

In one example, the system comprises about 1×10⁵ to about 1×10¹⁰ CFU probiotic bacteria, or about 1×10⁶ to about 1×10⁹ CFU probiotic bacteria, or about 1×10⁵ to about 1×10⁷ CFU probiotic bacteria, about 1×10⁶ to about 1×10⁸ CFU probiotic bacteria, about 1×10⁷ to about 1×10⁹ CFU probiotic bacteria, about 1×10⁸ to about 1×10¹⁰ CFU probiotic bacteria, or about 1×10⁵ to about 5×10⁵ CFU, 5×10⁵ to about 1×10⁶ CFU, about 1×10⁶ to about 5×10⁶ CFU, 5×10⁶ to about 1×10⁷ CFU, about 1×10⁷ to about 5×10⁷ CFU, 5×10⁷ to about 1×10⁸ CFU, about 1×10⁸ to about 5×10⁸ CFU, 5×10⁸ to about 1×10⁹ CFU, about 1×10⁹ to about 5×10⁹ CFU, 5×10⁹ to about 1×10¹⁰ CFU, or about 1.0×10⁵ CFU, about 1.5×10⁵ CFU, about 2.0×10⁵ CFU, about 2.5×10⁵ CFU, about 3.0×10⁵ CFU, about 3.5×10⁵ CFU, about 4.0×10⁵ CFU, about 4.5×10⁵ CFU, about 5.0×10⁵ CFU, about 5.5×10⁵ CFU, about 6.0×10⁵ CFU, about 6.5×10⁵ CFU, about 7.0×10⁵ CFU, about 7.5×10⁵ CFU, about 8.0×10⁵ CFU, about 8.5×10⁵ CFU, about 9.0×10⁵ CFU, about 9.5×10⁵ CFU, about 1.0×10⁶ CFU, about 1.5×10⁶ CFU, about 2.0×10⁶ CFU, about 2.5×10⁶ CFU, about 3.0×10⁶ CFU, about 3.5×10⁶ CFU, about 4.0×10⁶ CFU, about 4.5×10⁶ CFU, about 5.0×10⁶ CFU, about 5.5×10⁶ CFU, about 6.0×10⁶ CFU, about 6.5×10⁶ CFU, about 7.0×10⁶ CFU, about 7.5×10⁶ CFU, about 8.0×10⁶ CFU, about 8.5×10⁶ CFU, about 9.0×10⁶ CFU, about 9.5×10⁶ CFU, about 1.0×10⁷ CFU, about 1.5×10⁷ CFU, about 2.0×10⁷ CFU, about 2.5×10⁷ CFU, about 3.0×10⁷ CFU, about 3.5×10⁷ CFU, about 4.0×10⁷ CFU, about 4.5×10⁷ CFU, about 5.0×10⁷ CFU, about 5.5×10⁷ CFU, about 6.0×10⁷ CFU, about 6.5×10⁷ CFU, about 7.0×10⁷ CFU, about 7.5×10⁷ CFU, about 8.0×10⁷ CFU, about 8.5×10⁷ CFU, about 9.0×10⁷ CFU, about 9.5×10⁷ CFU, about 1.0×10⁸ CFU, about 1.5×10⁸ CFU, about 2.0×10⁸ CFU, about 2.5×10⁸ CFU, about 3.0×10⁸ CFU, about 3.5×10⁸ CFU, about 4.0×10⁸ CFU, about 4.5×10⁸ CFU, about 5.0×10⁸ CFU, about 5.5×10⁸ CFU, about 6.0×10⁸ CFU, about 6.5×10⁸ CFU, about 7.0×10⁸ CFU, about 7.5×10⁸ CFU, about 8.0×10⁸ CFU, about 8.5×10⁸ CFU, about 9.0×10⁸ CFU, about 9.5×10⁸ CFU, about 1×10⁹ CFU, about 1.5×10⁹ CFU, about 2.0×10⁹ CFU, about 2.5×10⁹ CFU, about 3.0×10⁹ CFU, about 3.5×10⁹ CFU, about 4.0×10⁹ CFU, about 4.5×10⁹ CFU, about 5.0×10⁹ CFU, about 5.5×10⁹ CFU, about 6.0×10⁹ CFU, about 6.5×10⁹ CFU, about 7.0×10⁹ CFU, about 7.5×10⁹ CFU, about 8.0×10⁹ CFU, about 8.5×10⁹ CFU, about 9.0×10⁹ CFU, about 9.5×10⁹ CFU, or about 1.0×10¹⁰ CFU probiotic bacteria. In another example, the system comprises about 1×10⁷ CFU probiotic bacteria.

The amount of artificial enzyme and linker in the system can be from about 10 μg to about 10 mg. In one example, the amount of artificial enzyme and linker in the system can be from about 10 μg to about 1 mg, about 1 mg to about 2 mg, about 2 mg to about 3 mg, about 3 mg to about 4 mg, about 4 mg to about 5 mg, about 5 mg to about 6 mg, about 6 mg to about 7 mg, about 7 mg to about 8 mg, about 8 mg to about 9 mg, about 9 mg to about 10 mg, or about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, or about 10.0 mg. In another example, the amount of artificial enzyme and linker in the system is about 10 μg to about 500 μg. In another example, the amount of artificial enzyme and linker in the system is about 50 μg.

The amount of artificial enzyme and linker used can be correlated to the number of bacteria used in the system. In one example, for every about 1×10⁷ CFU probiotic bacteria used in the system, the amount of artificial enzyme and linker comprises about 10 μg to about 1000 μg, or about 10 μg to about 500 μg, or about 10 μg to about 200 μg, or about 100 μg to about 300 μg, about 200 μg to about 400 μg, about 300 μg to about 500 μg, about 400 μg to about 600 μg, about 500 μg to about 700 μg, about 600 μg to about 800 μg, about 700 μg to about 900 μg, about 800 μg to about 1000 μg, or about 10 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 550 μg, about 600 μg, about 650 μg, about, 700 μg, about 750 μg, about 800 μg, about 850 μg, about 900 μg, about 950 μg, or about 1000 μg. In another example, for every about 1×10⁷ CFU probiotic bacteria used in the system, the amount of artificial enzyme and linker is about 50 μg.

The system described herein is manufactured by combining the artificial enzyme that facilitates a redox reaction, the probiotic bacteria and the linker, wherein the linker is responsible for conjugating the artificial enzyme and probiotic bacteria. In one example, there is provided a method of manufacturing the system as disclosed herein, comprising incubating (i) an artificial enzyme conjugated to a linker, wherein the artificial enzyme facilitates a redox reaction; and (ii) a probiotic bacteria, wherein the linker conjugates the artificial enzyme and probiotic bacteria. The linker is first conjugated to the artificial enzyme by a first incubation step, comprising incubating the artificial enzyme and the linker. The first incubation step can be, for example, about 1 hour, about 2 hours, about 3 hours, about 4 hours, about 5 hours, about 6 hours, about 7 hours, about 8 hours, about 9 hours, or about 10 hours. After the first incubation step, the artificial enzyme and linker is incubated with the probiotic bacteria in a second incubation step. The second incubation step can be, for example, about 10 minutes, about 20 minutes, about 30 minutes, about 40 minutes, about 50 minutes, or about 60 minutes. This second incubation step allows the probiotic bacteria to be bind to the artificial enzyme and the linker via conjugation through the linker. The method of incubation is generally known in the art.

In another example, the linker is first conjugated to the probiotic bacteria by a first incubation step, comprising incubating the probiotic bacteria and the linker. After the first incubation step, the probiotic bacteria and linker is incubated with the artificial enzyme in a second incubation step. In another example, the linker is conjugated to the artificial enzyme and probiotic bacteria at the same time.

The system described herein can be formulated into compositions, for example pharmaceutical compositions, suitable for administration. Where applicable, the system may be administered with a pharmaceutically acceptable carrier. A “carrier” can include any pharmaceutically acceptable carrier as long as the carrier can is compatible with other ingredients of the formulation and not injurious to the patient. Accordingly, pharmaceutical compositions for use may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Thus, in one example, the present disclosure describes a pharmaceutical composition comprising, but not limited to, the system described herein. In one example, the pharmaceutical composition comprises the system described herein. In yet another example, the pharmaceutical composition further comprises one or more pharmaceutically acceptable excipients, vehicles or carriers. In another example, the pharmaceutical composition further comprises a pharmaceutically acceptable carrier. Therefore, in one example, the pharmaceutical composition may further comprise a compound that can be, but not limited to, a pharmaceutically acceptable carrier, a liposomal carrier, an excipient, an adjuvant or combinations thereof.

The composition, shape, and type of dosage forms of the system described herein will typically vary depending on the intended use. For example, a dosage form used in the acute treatment of a disease or a related disease may contain larger amounts of one or more of the active compound it comprises than a dosage form used in the chronic treatment of the same disease. Similarly, a parenteral dosage form may contain smaller amounts of one or more of the active compound it comprises than an oral dosage form used to treat the same disease or disorder. These and other ways in which specific dosage forms encompassed by this invention will vary from one another will be readily apparent to those skilled in the art. Examples of dosage forms include, but are not limited to: tablets; caplets; capsules, such as soft elastic gelatine capsules; cachets; troches; lozenges; dispersions; suppositories; ointments; cataplasms (poultices); pastes; powders; dressings; plasters; solutions; gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms particularly suitable for parenteral administration to a patient; and sterile solids (e.g., crystalline or amorphous solids) that can be reconstituted to provide liquid dosage forms suitable for parenteral administration to a patient. Thus, in one example, the system described herein is provided in a form that can be, but not limited to, tablets, caplets, capsules, hard capsules, soft capsules, soft clastic gelatine capsules, hard gelatine capsules, cachets, troches, lozenges, dispersions, suppositories, ointments, cataplasms, poultices, pastes, powders, dressings, plasters, solutions, gels, suspensions, aqueous liquid suspensions, non-aqueous liquid suspensions, oil-in-water emulsions, a water-in-oil liquid emulsions, solutions, sterile solids, crystalline solids, amorphous solids, solids for reconstitution or combinations thereof.

The composition can be administered to the subject in any suitable way, including: orally, parenterally, topically, rectally, nasally or buccally. The term “parenteral” as used herein includes intravenous or intracranial injection.

The pharmaceutically acceptable carrier can comprise any suitable diluent, adjuvant, excipient, buffer, stabilizer, isotonicising agent, preservative or antioxidant. It will be appreciated that the pharmaceutically acceptable carrier should be non-toxic and should not interfere with the efficacy of the system or pharmaceutical composition described herein. The precise nature of the carrier or any other additive to the composition will depend on the route of administration and the type of treatment required. Pharmaceutical compositions can be produced, for instance, by means of conventional mixing, dissolving, granulating, dragee-making, levigating, emulsifying, encapsulating, entrapping or lyophilizing processes.

Orally acceptable dosage forms of the composition include, but are not limited to, capsules, tablets, pills, powders, liposomes, granules, spheres, dragees, liquids, gels, syrups, slurries, suspensions and the like. Suitable oral forms will be known to those of skill in the art. A tablet can include a solid carrier such as gelatine or an adjuvant or an inert diluent. Liquid pharmaceutical compositions generally include a liquid carrier such as water, petroleum, animal or vegetable oils, a mineral oil or a synthetic oil. Physiological saline solution, or glycols such as ethylene glycol, propylene glycol or polyethylene glycol may be included. Such compositions and preparations will generally contain at least 0.1 wt % of the system or pharmaceutical composition described herein and, in one example, up to about 25 wt %, depending on its solubility in the given carrier.

The composition can include a delivery vehicle for delivering the compound to a particular organ or tissue and/or for ensuring that the compound is able to be, for instance, ingested through the gut without loss of biological efficacy. Delivery vehicles can comprise, for example, lipids, polymers, liposomes, emulsions, antibodies and/or proteins. Liposomes are particularly preferred for delivering the compound through the skin. In one example, the liposome is a fluorescent liposome that comprises the system described herein. The fluorescent liposome is produced by introducing one or more fluorescent dyes during the production of the liposome that comprises the system described herein. The fluorescent dye can be, but is not limited to DiIC18 (7) (1,1′-Dioctadecyl-3,3,3′,3′-Tetramethylindotricarbocyanine Iodide (DIC), DiR Iodide, IR800, BODIY, IR808 and Cy5.5, or any combination thereof.

The composition can be delivered using a sustained-release system, such as semipermeable matrices of solid hydrophobic polymers containing the compound. Various sustained-release materials are available and well known by those skilled in the art. Sustained-release capsules may, depending on their chemical nature, release the compound for about 1 to 20 weeks.

Inflammation is a complex process that implicates many different players such as proinflammatory factors and anti-inflammatory factors. It is envisaged that the system and pharmaceutical composition described herein are capable of reducing any inflammatory burden and disorders by decreasing the levels of proinflammatory factors or increasing the levels of anti-inflammatory factors. In one aspect, there is provided a method of decreasing the levels of proinflammatory factors by administering a therapeutically effective amount of the system described herein or the pharmaceutical composition described herein. In one example, the system as disclosed herein or the pharmaceutical composition described herein is for use in decreasing the levels of proinflammatory factors in a subject thereof. For example, the proinflammatory factors comprises, but are not limited to, myeloperoxidase (MPO), interleukin 6 (IL-6), tumor-necrosis factor alpha (TNF-α), interferon gamma (IFN-γ) and CD45.

In another aspect, there is provided a method of increasing the levels of anti-inflammatory factors by administering a therapeutically effective amount of the system described herein or the pharmaceutical composition as disclosed herein. In one example, the system described herein or the pharmaceutical composition described herein is for use in increasing the levels of anti-inflammatory factors in a subject thereof. For example, the anti-inflammatory factors comprise, but are not limited to, interleukin 10 (IL-10) or transforming growth factor β (TGF-β).

It is also envisaged that the system described herein or the pharmaceutical composition described herein is for use in therapy. Accordingly, the system and pharmaceutical composition described herein may be incorporated in treatment tools, treatment regimens or methods of treatment. As used herein, the term “treatment” refers to any and all uses which remedy a disease state or symptoms, prevent the establishment of disease, or otherwise prevent, hinder, retard, or reverse the progression of disease or other undesirable symptoms in any way whatsoever.

In one aspect, there is provided a method of improving gastrointestinal tract health by improving microbial balance in a subject in need thereof by administering a therapeutically effective amount of the system described herein or the pharmaceutical composition described herein. In one example, the system described herein or the pharmaceutical composition described herein are for use in improving gastrointestinal tract health by improving microbial balance. In another example, there is provided a use of the system described herein or the pharmaceutical composition described herein in the manufacture of a medicament for improving gastrointestinal tract health by improving microbial balance. As used herein, the term “gastrointestinal tract” refers to the organs by which food and water enters, are digested and/or absorbed and expelled. The organs include, but are not limited to, the small intestine, large intestine, stomach, mouth, pharynx, esophagus, rectum, and anus.

A disruption in the microbial balance can result in dysbiosis, which as used herein refers to an imbalance in the microbiota in the gastrointestinal tract, resulting in changes to the functional composition and metabolic activities. Examples of the microbiota imbalance refers to the reduction of probiotic bacteria population and/or the increment of potentially harmful bacteria population. Dysbiosis is a gastrointestinal tract disorder and can be characterized by symptoms such as, but not limited to bloating, flatus, spasms, stomach upset. Chronic dysbiosis can also lead to more severe gastrointestinal tract disorders. For example, irritable bowel syndrome patients are shown to have dysbiosis characterized by a lower proportion of Firmicutes and Firmicutes/Bacteroidetes ratio, and an increase in Verrucomicrobiota.

In another aspect, there is provided a method of treating a disease in a subject in need thereof by administering a therapeutically effective amount of the system described herein or the pharmaceutical composition described herein. As used herein, the term “treat” or “treating” is intended to refer to providing an pharmaceutically effective amount of the system or the respective pharmaceutical composition or medicament thereof, sufficient to act prophylactically to prevent the development of a weakened and/or unhealthy state; and/or providing a subject with a sufficient amount of the complex or pharmaceutical composition or medicament thereof so as to alleviate or eliminate a disease state and/or the symptoms of a disease state, and a weakened and/or unhealthy state. In one example, the disease is a gastrointestinal tract disorder. In another example, the system described herein or the pharmaceutical composition described herein are for use in treating a gastrointestinal tract disorder. In another example, there is provided a use of the system described herein or the pharmaceutical composition described herein in the manufacture of a medicament for treating a gastrointestinal tract disorder in a subject in need thereof. For example, the gastrointestinal tract disorder is, but are not limited to, inflammatory bowel disease (IBD) of ulcerative colitis, inflammatory bowel disease (IBD) of Crohn's disease, dysbiosis, necrotizing enterocolitis, acute infectious diarrhea, antibiotic-associated diarrhea, infant colic.

The system or the pharmaceutical composition thereof can be administered to a subject in need thereof. The term “subject” as used herein refers to an animal, preferably a mammal or a bird, who is the object of administration, treatment, observation or experiment. “Mammal” includes humans and both domestic animals such as laboratory animals and household pets, (e.g. cat, dog, swine, cattle, sheep, goat, horse, rabbit), and non-domestic animals such as wildlife, fowl, birds and the like. More particularly, the mammal is a rodent (e.g. mouse, rat, guinea pig, hamster, squirrel). Still, most particularly, the mammal is a human.

The system or the pharmaceutical composition thereof can be administered to the subject in either a prophylactically effective or a therapeutically effective amount as necessary for the diseases described herein. The actual amount of the composition and rate and time-course of administration of the composition, will depend on the nature and severity of the gastrointestinal tract disorder being treated or the prophylaxis required. Prescription of treatment such as decisions on dosage and the like will be within the skill of the medical practitioner or veterinarian responsible for the care of the subject. Typically, the system or the pharmaceutical composition for administration to a subject will comprise about 1.0×10⁷ to about 3.0×10⁸ CFU probiotic bacteria per kg of body weight and about 1.0 mg to about 3.0 mg artificial enzyme and linker per kg of body weight. It will be readily understood that the unit “CFU/kg” refers to number of probiotic bacteria per kg of body weight. In one example, the unit “CFU/kg” refers to the number of Bifidobacterium longum that is orally administered per kg body weight. The unit “mg/kg” refers to the amount of artificial enzyme and linker per kg of body weight. In one example, the system or the pharmaceutical composition to be administered comprises about 1.0×10⁷ to about 3.0×10⁸ CFU/kg probiotic bacteria; and about 1.0 to about 3.0 mg/kg artificial enzyme and linker. In another examples, the system or the pharmaceutical composition to be administered comprises about 1.0×10⁷ CFU/kg, about 1.5×10⁷ CFU/kg, about 2.0×10⁷ CFU/kg, about 2.5×10⁷ CFU/kg, about 3.0×10⁷ CFU/kg, about 3.5×10⁷ CFU/kg, about 4.0×10⁷ CFU/kg, about 4.5×10⁷ CFU/kg, about 5.0×10⁷ CFU/kg, about 5.5×10⁷ CFU/kg, about 6.0×10⁷ CFU/kg, about 6.5×10⁷ CFU/kg, about 7.0×10⁷ CFU/kg, about 7.5×10⁷ CFU/kg, about 8.0×10⁷ CFU/kg, about 8.5×10⁷ CFU/kg, about 9.0×10⁷ CFU/kg, about 9.5×10⁷ CFU/kg, about 1.0×10⁸ CFU/kg, about 1.5×10⁸ CFU/kg, about 2.0×10⁸ CFU/kg, about 2.5×10⁸ CFU/kg, or about 3.0×10⁸ CFU/kg probiotic bacteria; and about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, or about 3.0 mg/kg artificial enzyme and linker, or any combination thereof. In another example, the system or the pharmaceutical composition to be administered comprises about 2.5×10⁸ CFU/kg probiotic bacteria; and about 2.5 mg/kg artificial enzyme and linker. In another example, the system or the pharmaceutical composition to be administered comprises about 2.5×10⁸ CFU/kg probiotic bacteria; and about 1.25 mg/kg artificial enzyme and linker.

As used in this application, the singular form “a,” “an,” and “the” include plural references unless the context clearly dictates otherwise. For example, the term “a genetic marker” includes a plurality of genetic markers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Material and Methods

### Chemicals

Zinc nitrate hexahydrate (Zn(NO₃)₂·6H₂O) and methanol were obtained from Beijing Chemicals (Beijing, China). Iron (III) 2,4-pentanedionate (Fe(acac)₃) and 2-methyl imidazole (2-MI) were ordered from Sigma-Aldrich (St. Louis, MO, USA). C12-PEG-B were acquired from Shaoxing Qixin Trading Co., LTD (Shaoxing, China). Bifidobacterium longum (BL, ATCC® BAA999™) was purchased from American Type Culture Collection.

### Synthesis of Single-Atom Nanozymes (Fe SA)

Single-atom nanozymes (Fe SA) were prepared by the pyrolysis of Fc(acac)_ENREF_2₃: Fe(acac)₃-encapsulated metal organic framework (MOF) (Fe@MOF. also referred to as FE@ZIF-8) in an inert nitrogen (N₂) environment. 30 mL homogeneous methanol solution of Zn(NO₃)₂·6H₂O (1160 mg) and Fe(acac)_ENREF_23: Fc(acac)₃ (35 mg) was mixed with 30 mL 2-methyl imidazole (2-MI) (1314 mg) methanol solution under mild stirring for 2 hours. Then, Fe@MOF was collected by centrifugation, rinsed and dried in a vacuum oven at 70° C. overnight. Finally, the dried Fe@MOF obtained was ground into powder and loaded into a porcelain boat for pyrolysis under 900° C. for 3 hours with a heating rate of 5° C./min. The yielded Fe SA was collected after cooling.

### Synthesis of C18-PEG-B Modified Single-Atom Catalyst (B-SA)

100 mL homogeneous solution of SA (1 mg/mL) and linker boronic acid-C18-poly(ethylene glycol) (C18-PEG-B) (1 mg/mL) were stirred under dark for 4 hours to obtain C18-PEG-B modified single-atom catalyst (B-SA). B-SA was collected by centrifugation, rinsed and re-dispersed in 100 mL phosphate-buffered saline (PBS) and stored in a dark place at 4° C.

### Synthesis of the Artificial Enzyme-Armed Probiotics (BL@B-SA)

Bifidobacterium longum (BL) were anaerobically cultured in a tube of #1490 broth under shaking at 37° C. for 18 hours. BL was incubated on brucella blood plate anaerobically at 37° C. to check for contamination and concentration. The artificial enzymes-probiotics platform of BL@B-SA₅₀ were prepared by incubating equivalent of B-SA (500 μg/mL) with BL (1×10⁸ CFU/mL) under shaking. After 30 minutes, the mixture was centrifuged, washed, redispersed into an anaerobic tube containing medium. The resulting BL@B-SA₅₀ was stored in a dark place at 4° C. BL@B-SA, with different ratios were synthesized by changing the concentrations of BL and B-SA.

### Synthesis of Fluorescent C18-PEG-B Modified Liposome (DIR-B-Lip)

The phospholipid ethanol solution (10 mg/mL) was first prepared by mixing lecithin (36.27 mg), cholesterol (5 mg), and 1, 2-Distearoyl-sn-glycero-3-phosphoethanolamine-Poly(ethylene glycol) (DSPE-PEG) (7.76 mg) in 4.9 mL ethanol. 1.08 mL phospholipid ethanol solution (10 mg/mL), 0.3 mL C18-PEG-B ethanol solution (4 mg/mL) and 0.12 mL DiIC18 (7); 1,1′-dioctadecyl-3,3,3′,3′-tetramethylindotricarbocyanine iodide (DIR) ethanol solution (5 mg/mL) were added into 4.5 mL chloroform in a round-bottom flask, wherein chloroform was allowed to evaporate under low pressure, resulting in the formation of a transparent film on the flask bottom. The film was hydrated with 12 mL PBS and sonicated to form a clear suspension of the fluorescent C18-PEG-B modified liposome (DIR-B-Lip).

### Synthesis of Fluorescent Liposome-Probiotics System (BL@DIR-B-Lip)

The fluorescent liposome-probiotics system (BL@DIR-B-Lip) was prepared via a similar process when synthesizing BL@B-SA₅₀, except that the B-SA was replaced by DIR-B-Lip

### Bacterial Viability Under Oxidative Stress

Native BL (1×10⁷ CFU/mL BL), BL@B-SA_{3.125} (1×10⁷ CFU/mL BL and 3.125 μg/mL B-SA), BL@B-SA_{6.25} (1×10⁷ CFU/mL BL and 6.25 μg/mL B-SA), BL@B-SA_{12.5} (1×10⁷ CFU/mL BL and 12.5 μg/mL B-SA), BL@B-SA₂₅ (1×10⁷ CFU/mL BL and 25 μg/mL B-SA), BL@B-SA₅₀ (1×10⁷ CFU/mL BL and 50 μg/mL B-SA) and BL@B-SA₁₀₀ (1×10⁷ CFU/mL BL and 100 μg/mL B-SA) were incubated with PBS buffer (pH 7.4, 10 mM) containing hydrogen peroxide (H₂O₂) (200 μM) at 37° C. for 2 hours before viability analysis, respectively. Bacterial viability was monitored via blood plate counting. Native BL (1×10⁷ CFU/mL BL) without H₂O₂ treatment was used as control and their viability levels were defined as 100%.

### Evaluation of the Growth Curves of BL and BL@B-SA

The growth curves of native BL and BL@B-SA under the anaerobic condition were tested. Specifically, 200 μL native BL (1×10⁷ CFU/mL BL) and BL@B-SA₅₀ (1×10⁷ CFU/mL BL and 50 μg/mL B-SA) were injected into tubes of 10 mL #1490 broth, respectively. After incubation for 12, 24, 36 and 48 hours, 100 μL mixture were collected from each tube and the numbers of bacteria were determined by blood plate counting methods.

### In Vitro ROS Scavenging Activities of BL@B-SA

For the protection of cells from H₂O₂ induced oxidative stress, different concentrations of BL@B-SA₅₀ were cultured with HT-29 cells in the absence or presence of H₂O₂ (200 μM) for 24 hours. Relative cell viabilities were tested by the 3-(4,5-dimethylthiazol-2-yl)-2,5-diphenyltetrazolium bromide (MTT) assay.

For confocal imaging and flow cytometry assays, HT-29 cells were cultured with BL@B-SA₅₀ (0, 2.5, 5 and 10 μg/mL) in the absence or presence of H₂O₂ (400 μM). After 2 hours, 20 μM 2′-7′dichlorofluorescein diacetate (DCFH-DA) was added to detect the intercellular ROS generation. After being washed with PBS three times, cells were qualitatively observed under confocal microscopy and the reactive oxygen species (ROS) contents were quantitatively analyzed by flow cytometry.

### Comparison of In Vitro ROS-Scavenging Abilities

The in vitro ROS-scavenging activities of BL, B-SA, BL+B-SA, BL@B-SA₅₀ were evaluated in equal concentrations by MTT assays, confocal microscopy and flow cytometry. The concentrations of nanomaterials in different measurements of MTT assays were 0.625 μg/mL while that for confocal microscopy and flow cytometry were both 2.5 μg/mL. The viabilities of HT-29 cells without treatment were defined as 100%, while the ROS content of HT-29 cells without treatment was defined as 1.

### Catalytic Activity and Bacterial Viability of BL@B-SA in the Gastrointestinal Tract

In order to investigate the stability of BL@B-SA in the gastrointestinal tract, 300 μL equal amount of BL (1×10⁷ CFU) and BL@B-SA₅₀ (containing 1×10⁷ CFU BL and 50 μg B-SA) were added into 700 μL medium supplemented with either simulated gastric fluid (SGF) containing pepsin (pH 1.2), simulated intestinal fluid (SIF) containing trypsin (pH 6.8) or simulated inflammatory colon fluid (SICF) containing 200 μM H₂O₂ (pH 7.8), and incubated at 37° C. with gentle shaking. At predetermined time points, both the catalytic activity and bacterial viability of BL@B-SA were measured.

To assess catalytic activities, the samples were centrifuged, washed and resuspended in PBS to obtain 500 μg/mL BL@B-SA₅₀ or BL. Using a final concentration of 50 μg/mL, the ROS-scavenging abilities were determined by Total Superoxide Dismutase Kit with WST-8, Catalase Assay Kit and TA probe.

To determine bacterial viability, 100 μl of diluted sample was taken and spread on blood plates. The colonies were counted after 48 hours incubation at 37° C.

### In Vivo Targeting

To demonstrate that probiotics can promote the targeting and colonization of the biocompatible artificial enzymes, photoacoustic (PA) imaging was carried out. 500 μL of B-SA or BL@B-SA₅₀ (each containing 500 μg/mL B-SA) was orally administered to the ulcerative colitis (UC) mice. After 6 hours, the mice were anesthetized with isoflurane and maintained at 37° C. Images were obtained in the supine position with the Vevo LAZR system and the images were collected at 808 nm.

To further verify that probiotics could promote the retention of nanomaterials, fluorescent DiR—B-Lip and BL@DiR—B-Lip were employed to replace B-SA and BL@B-SA₅₀. After 6 hours, ulcerative colitis (UC) mice were euthanized and intestines were excised. Fluorescence intensities in intestines from each group were analyzed using IVIS Lumina Series III in vivo imaging system with a Cy5.5 filter channel and an exposure time of 1 second.

### Dextran Sulfate Sodium (DSS)-Induced Ulcerative Colitis (UC) of Mice

Six-week-old female C57BL/6 mice were housed in groups of five mice per cage and acclimatized for 1 week before inclusion in the study. Mice received 3% Dextran sulfate sodium (DSS) supplemented in the drinking water for 4 days to induce ulcerative colitis (UC) model, followed by normal water. Control healthy mice were provided with normal water only. Then different ratio of BL@B-SAₓ (BL@B-SA₂₅, BL@B-SA₅₀, BL@B-SA₁₀₀), 1.25 mg/kg B-SA, BL, BL@B-SA₅₀, BL+B-SA mixture (equivalent dose of BL and B-SA of BL@B-SA₅₀), 1 mg/kg of MPS, 1 mg/kg of DEX, 40 mg/kg of 5-ASA or PBS, as well as 0.625, 1.25, 2.5 and 5 mg/kg BL@B-SA₅₀ were administered via an oral route into mice on predetermined days. Changes in bodyweight were assessed daily over the 9-day experimental period. Feces were collected on a predetermined day for microbiome analysis. On the last day of the experiment, mice were sacrificed, and the entire colon was excised. Colon length was measured and gently washed with physiological saline. Two pieces of colons were used for histological assessment and immunofluorescence staining. The remaining colon tissue samples were used for enzyme-linked immunosorbent assay (ELISA) analysis and reactive oxygen species (ROS) evaluation.

### 2,4,6-Trinitrobenzene Sulfonic Acid (TNBS)-Induced Chron's Disease (CD) of Mice

Female C57BL/6 mice were acclimatized for 7 days and randomly divided into various groups with five mice per group. The TNBS-induced mice were pre-sensitized with 1 wt % 2,4,6-Trinitrobenzene sulfonic acid (TNBS) and kept for 7 days. The mice were further treated with 2.5 wt % TNBS enema on day 8. Finally, B-SA, BL, BL+B-SA, and BL@B-SA were treated using the same protocol as the DSS model described above.

### Acetic Acid-Induced Ulcerative Colitis (UC) of Dogs

9 Beagle dogs were acclimatized for 1 week before inclusion in the study. Before building the UC model on dogs, the intestines of dogs were first cleaned using enema. After anesthesia, a polyethylene catheter was inserted 20 cm from the anus into the colon, and a 7% acetic acid solution (2 mL/kg) was slowly injected. Immediately after the injection, the head was kept low and the tail was kept high to allow the acetic acid to etch the colon to build the UC model. Finally, the colons of dogs were flushed twice with 100 mL physiological saline, and the dogs were allowed to lie still. The state and fecal traits of the dogs in each group were observed after waking up. After one day, the UC dogs were divided into two groups. The control group containing 3 UC dogs was given media, whereas the BL@B-SA group including 3 UC dogs was orally administered with BL@B-SA for 5 consecutive days. For the healthy group, 3 healthy dogs underwent a similar process as the control group except that 7% acetic acid was replaced by physiological saline and given media every day. Changes in bodyweight were assessed daily over the 8-day experiment period. On days 0, 1, 2, 4 and 7, the dogs were anesthetized for endoscopy. On the last day, the blood samples of dogs were collected to perform complete blood panel analysis, while the colon and major organs (heart, liver, spleen, lung and kidneys), as well as digestive tracts (esophagus, stomach, small intestine, jejunum and ileum), were excised for the histological assessment and immunofluorescence staining.

### EXPERIMENTAL RESULTS

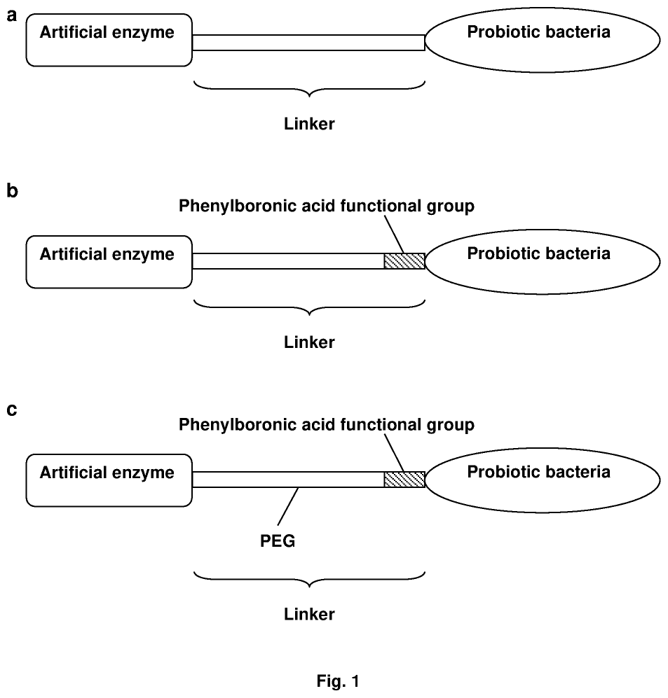

An artificial enzyme-probiotic bacteria system has been developed, in which the probiotic bacteria can promote the targeting and colonization of SAzymes to scavenge elevated reactive oxygen species (ROS) and inflammatory factors. This in turn alleviates the inflammation, thereby improving the bacterium viability to reshape the intestinal barrier functions and gut microbiota. This system can be applied to inflammatory bowel disease (IBD) treatment by combining Bifidobacterium longum (BL) and single atom nanozymes (SAzymes) via, a linker comprising a phenylboronic acid functional group, for example, a C18-PEG-B linker (FIG. 1c). The artificial enzyme-probiotic bacteria system developed shows that Bifidobacterium longum (BL) can improve the targeting and colonization of SAzymes to reduce inflammation, while SAzymes could mimic antioxidant enzymes to maintain high bacterium viability under the harsh oxidative conditions ( ). Ultimately, the resulting probiotics-SAzyme (BL@B-SA) system reduces excess ROS levels, inhibit proinflammatory cytokine production, restore the intestinal barrier functions as well as increase the richness and diversity of gut microbiota, exhibiting prominent in vivo therapeutic effect in ulcerative colitis (UC) and Chron's Disease (CD) models in mice as well as ulcerative colitis (UC) models in beagle dogs. This artificial enzyme-probiotic bacteria system presents a new strategy to treat inflammatory bowel disease (IBD), as well as provides new insight into disease treatment by integrating probiotics and artificial enzymes.

### Design and Characterization of BL@B-SA.

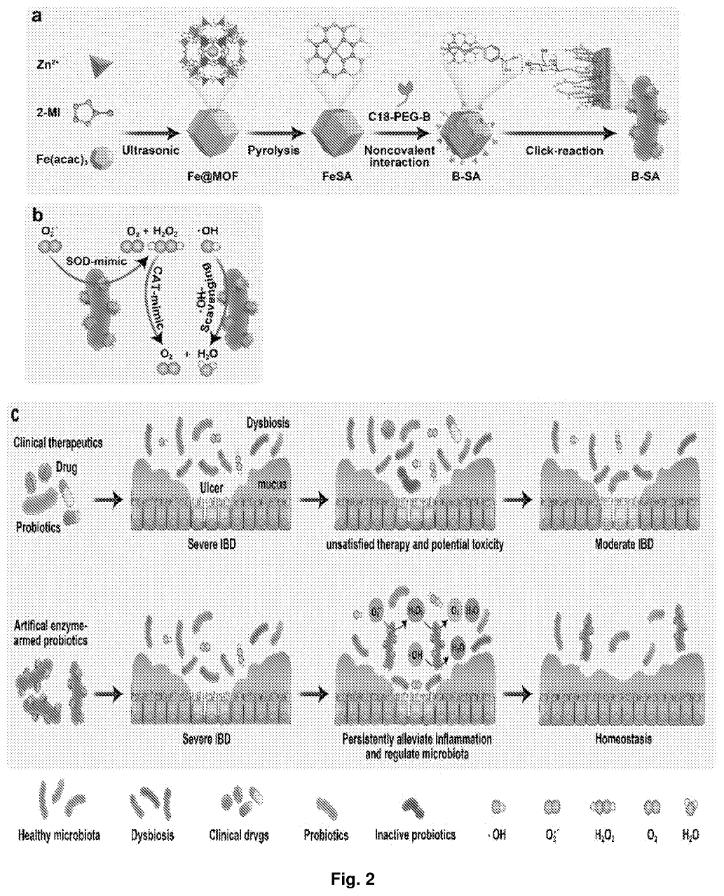

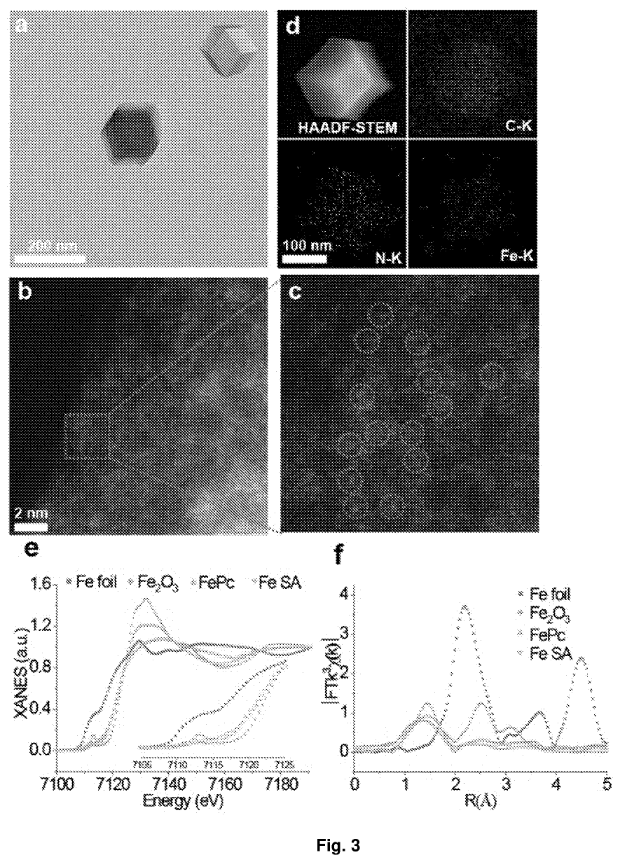

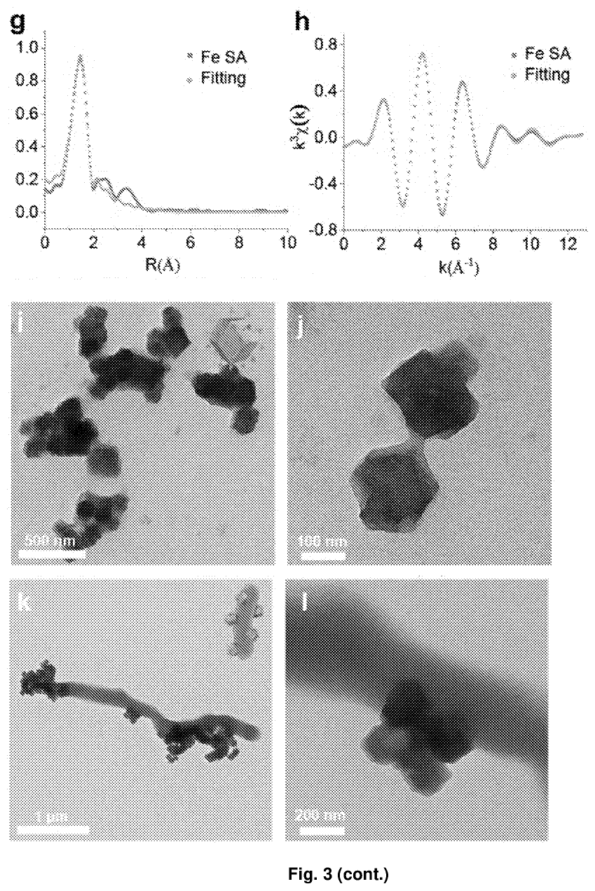

Single-atom nanozymes (Fe SA) was prepared through the pyrolysis of metal-organic framework encapsulated iron (Fe) precursors (Fe@MOF, also known as Fe@ZIF-8) (FIG. 2a), as characterized by scanning electron microscopy and transmission electron microscopy. The absence of iron-containing nanoparticles was revealed by the high-resolution transmission electron microscopy (HRTEM) images (FIG. 3a) and powder X-ray diffraction (XRD) test. Meanwhile, the atomically dispersed single Fe atom was confirmed by the aberration-corrected atomic-resolution high angle annular dark-field scanning transmission electron microscopy (HAADF-STEM) (FIGS. 3b and c) and energy dispersive spectra (EDS) (FIG. 3d). Further, the structure of Fe SA was analyzed by Raman spectroscopy and X-ray photoelectron spectroscopy (XPS) spectrum, suggesting the coordination of Fe—Nₓ and presence of graphitic carbon. Furthermore, the accurate structure of Fe atoms in the Fe SA was further disclosed by the synchrotron radiation-based X-ray absorption near-edge structure (XANES) and extended X-ray absorption fine structure (EXAFS) measurements. The positively charged Fe^{δ+} were stabilized by N atoms, and they were atomically dispersed throughout the whole Fe SA₃₈, as manifested in Fe K-edge XANES spectra (FIG. 3e) and Fourier-transformed κ³-weighted EXAFS spectra (FIG. 3f). Quantitatively, atomic Fe sites were verified to be five-coordinated by nitrogen species through the EXAFS fitting (FIG. 3g-h and Table 1), and the Fe atom loading was determined to be 1.31 wt % by the inductively coupled plasma mass spectrometry (ICP-MS).

TABLE 1
EXAFS fitting parameters at the Fe K-edge for Fe SA.

| Sample | Shell | N^{a} | R(Å)^{b} | σ² (Å².10³)^{c} | ΔE₀ (eV)^{d} | R factor |
|---|---|---|---|---|---|---|
| Fe SA | Fe—N | 5.0 | 2.00 | 9.0 | −0.5 | 0.4 |

N: coordination numbers; R: bond instance; σ²: Debye-Waller factors; ΔE₀: the inner potential correction. R factor: goodness of fit. S₀², 0.8, was obtained from the experimental EXFAS fit of Fe₂O₃. Error bounds that characterize the structural parameters obtained by EXAFS spectroscopy were estimated as N ± 20%; R ± 1%; σ² ± 20%; ΔE₀ ± 20.

To further integrate artificial enzymes with probiotics, the linker of boronic acid-poly(ethylene glycol) (C18-PEG-B) was employed. C18-PEG-B could not only reversibly bind polysaccharides from the bacterial cell wall via boronic acid vicinal-diol-based click reaction, but also be modified on the Fe SA surface through the noncovalent interactions of hydrophobic C18 group and graphitic carbon. B-SA was successfully fabricated by functionalizing Fe SA with C18-PEG-B, with the edges of Fe SA blurring (FIGS. 3i and j) and the zeta potential changing from negative to neutral. Finally, by simply mixing different proportions of B-SA with BL, the probiotics-artificial enzymes system of BL@B-SAₓ (x means the content of B-SA (in μg) relative to 1×10⁷ CFU BL) could be constructed, and can be used in vitro or in vivo. When used in vivo, the body weight of the subject is taken into consideration when calculating BL@B-SAₓ. For example, BL@B-SA₅₀ refers to 2.5×10⁸ CFU probiotic bacteria (for example, Bifidobacterium longum) per kg body weight and 1.25 mg artificial enzyme and linker (for example, B-SA) per kg body weight. This is equivalent to 50 μg artificial enzyme and linker in 1×10⁷ CFU probiotic bacteria. In another example, BL@B-SA 100 refers to 2.5×10⁸ CFU probiotic bacteria per kg and 2.5 mg artificial enzyme and linker per kg body weight.

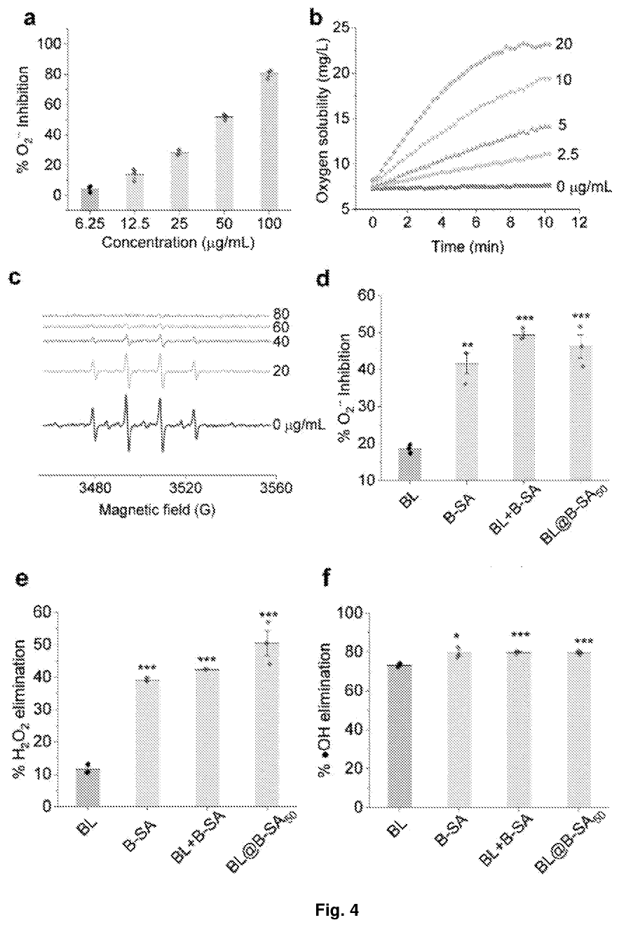

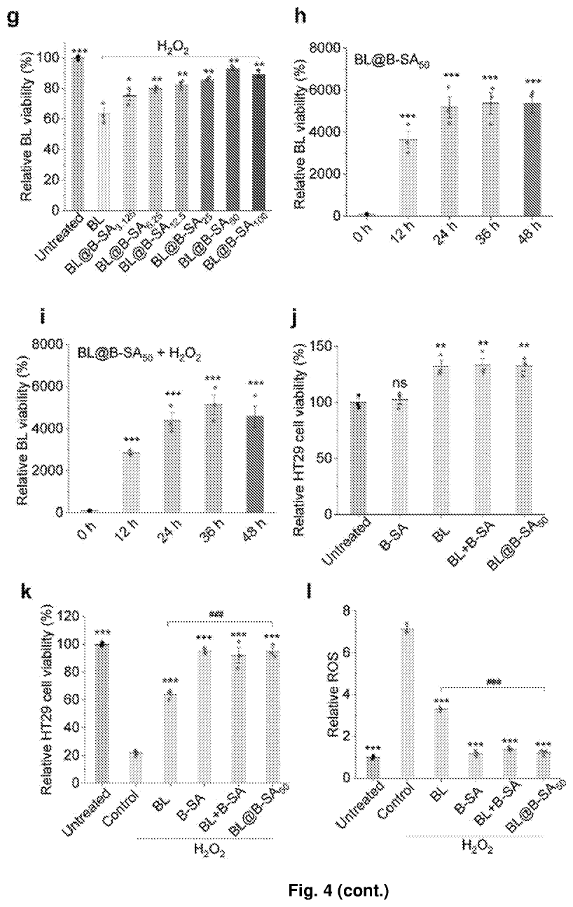

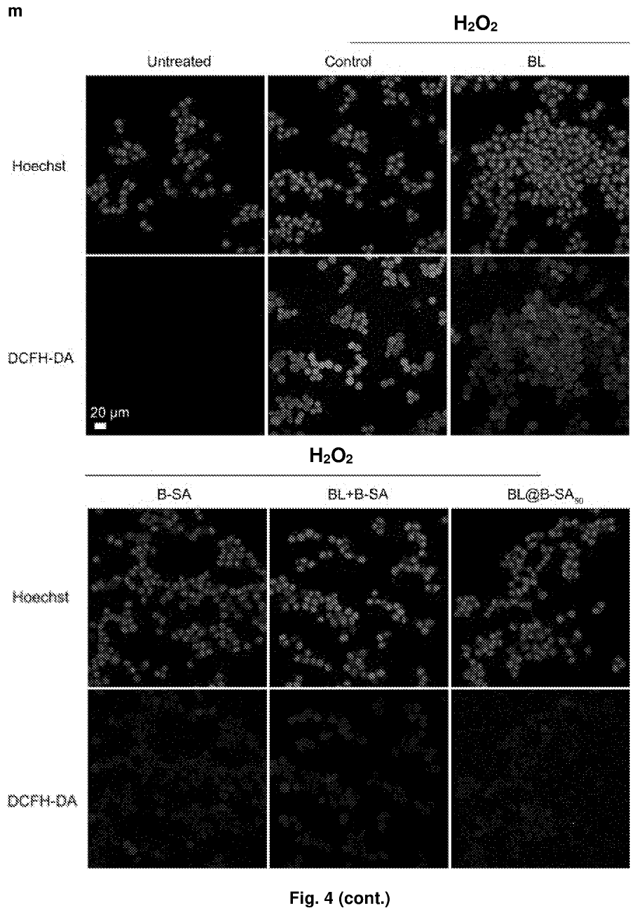

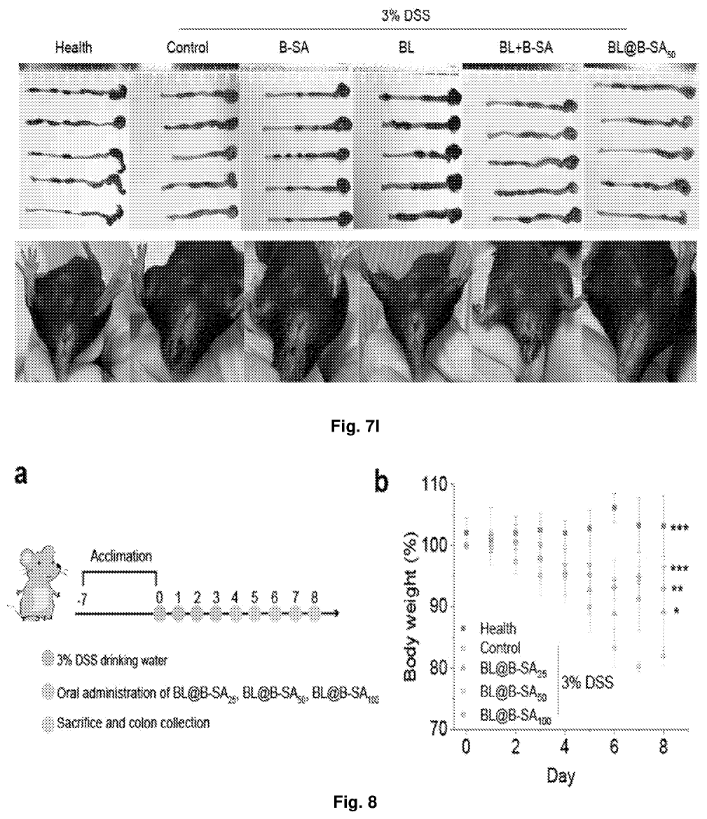

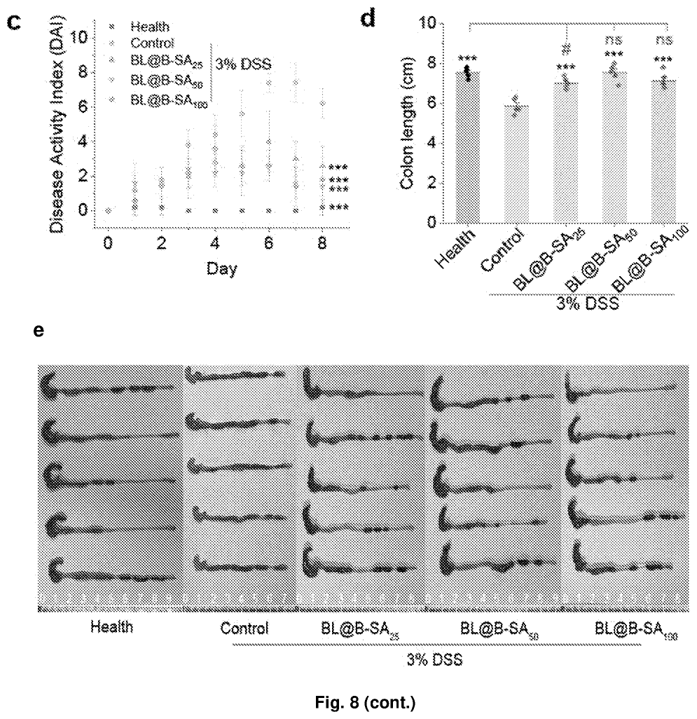

This is equivalent to 100 μg artificial enzyme and linker in 1×10⁷ CFU probiotic bacteria. As revealed by the TEM image, B-SA were positioned on the surface of BL, and no morphology change of BL occurred during this process (FIGS. 3k and l). BL@B-SA₅₀ was then investigated in detail to identify their optimal bacterial protecting ability and IBD therapeutic effects (FIG. 4g and FIG. 8).

### ROS-Scavenging Ability of BL@B-SA

The ROS-scavenging abilities of B-SA towards O₂″, H₂O₂, and ·OH, representative ROS involved in IBD, were monitored (FIG. 4a-c). First, the SOD-like activity for O₂″ elimination was investigated by employing an O₂″-specific Total Superoxide Dismutase Assay Kit with WST-8. As exhibited in FIG. 4a, B-SA possessed outstanding SOD-like activity by catalyzing the disproportionation of O₂″ to H₂O₂ and O₂. The produced H₂O₂ could be further eliminated through the CAT-like properties of B-SA, manifested by the generated O₂ (FIG. 4b) and the distinct reduction of H₂O₂ absorbance at 240 nm. The H₂O₂-scavenging ability was further quantified by Catalase Assay Kit. Besides, the ·OH-scavenging activity of B-SA was detected by 5,5′-dimethyl-1-pyrroline-N-oxide (DMPO) probe (FIG. 4c) and terephthalic acid (TA) probe, respectively. These distinctly decreased signal intensities affirmed that B-SA could effectively scavenge. OH. Overall, B-SA exhibited high scavenging activities against the three representative ROS and these performances intensified with the increasing concentration of B-SA, implying their potential to protect probiotics and cells from oxidative stress.

To reveal whether the integration of B-SA will endow Bifidobacterium longum (BL) with amplified antioxidant ability, the ROS-scavenging activities among bare BL, B-SA, BL+B-SA, and BL@B-SA₅₀ were next compared. By arming with B-SA, BL exhibited significantly enhanced ROS-scavenging activity over native BL (FIG. 4d-f), ensuring their tolerance in the hash oxidative microenvironment. The defense of B-SA enclosure against ROS was analyzed by feeding toxic H₂O₂ to engineered probiotics. Various concentrations of B-SA to BL significantly potentiated the bacterium viability, with BL@B-SA₅₀ achieving the best protecting effects (FIG. 4g). Then, the long-term fate of BL@B-SA₅₀ in the absence or presence of H₂O₂ was investigated. Surprisingly, engineering with B-SA not only unaltered the metabolism of BL under the normal conditions (FIG. 4h), but also protected their proliferation until reaching the stationary phase in H₂O₂ medium (FIG. 4i).

Having validated the cytoprotecting ability of artificial enzymes to probiotics, the in vitro antioxidant potential of the BL@B-SA₅₀ ensemble was further investigated by using the HT29 cell line as a model (BL, B-SA, BL+B-SA were also studied as a comparison). The cytotoxicity results demonstrated that single B-SA was nontoxic to HT29, facilitating the subsequent biological applications. Meanwhile, native BL could promote the growth of HT29, which might be attributed to the secreted bioactive factors (FIG. 4j). After combination, BL@B-SA₅₀ could still enhance the growth of HT29, promising to repair the intestinal mucosal tissue. When exposed to 200 μM H₂O₂, the viabilities of HT29 cells were sharply reduced to approximately 20% (FIG. 4k), indicating the distinct oxidative damage. However, under the protection of B-SA, BL+B-SA and BL@B-SA₅₀, the viabilities were significantly improved, attributing to the cytoprotecting ability of B-SA. Notably, the viability of the BL-treated group was also enhanced, owing to the antioxidant activity of exopolysaccharides (EPS) of BL. The antioxidant mechanisms were analyzed using flow cytometry and fluorescence microscopy. Obviously, ROS could be effectively scavenged by BL@B-SA₅₀, with higher efficacy than BL alone (FIGS. 4l and m). Taken together, the in vitro results demonstrated that BL@B-SA₅₀ could promote the growth of HT29 cells and protect them from ROS attack.

### Catalytic Activity and Bacterial Viability of BL@B-SA in the Gastrointestinal Tract

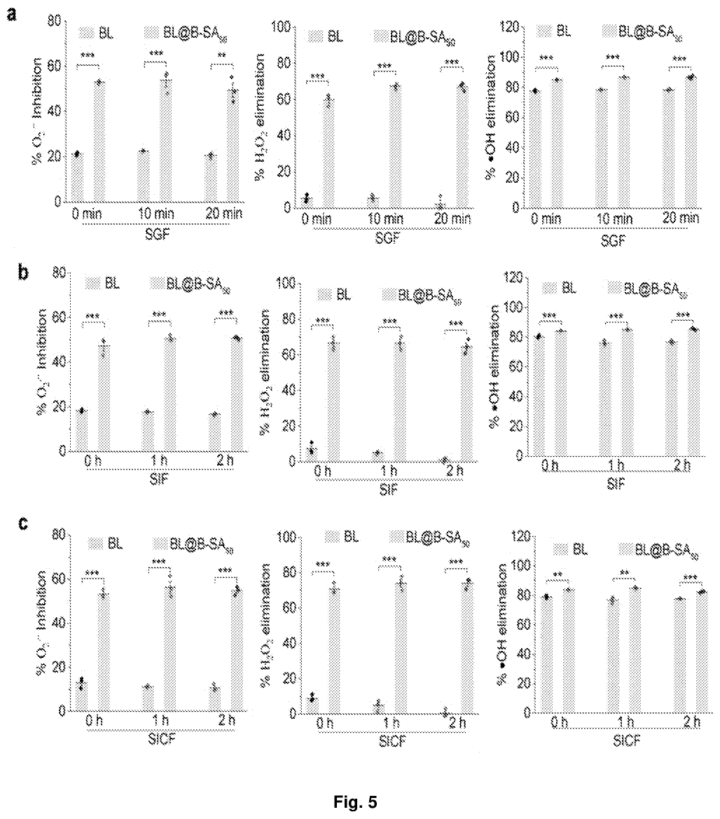

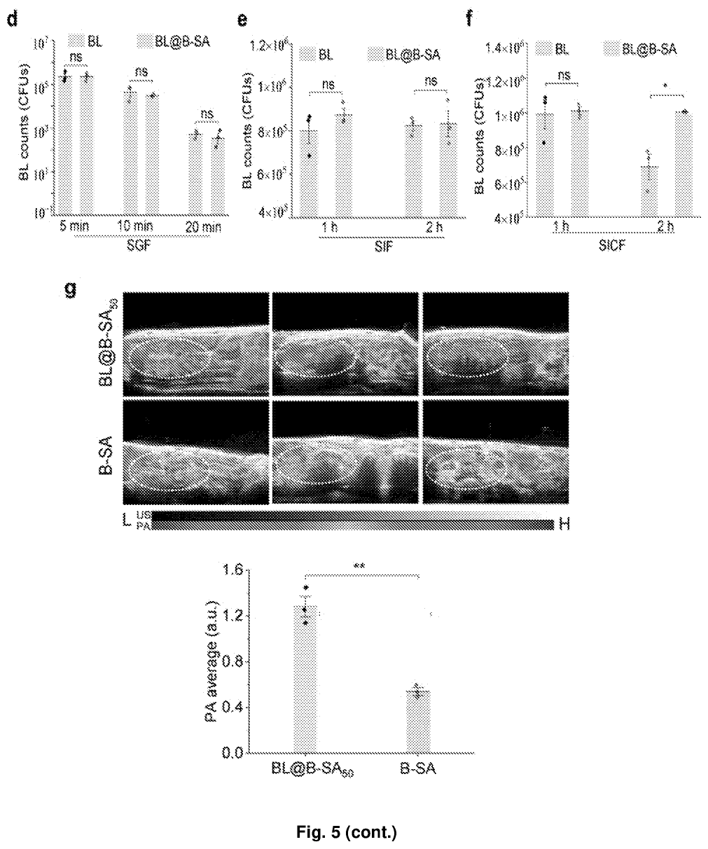

Before reaching the inflamed intestine, BL@B-SA will likely suffer from the harsh gastrointestinal environment after oral ingestion, such as gastric acid, bile salts and inflammation, which can destabilize artificial enzymes and even deactivate bacteria. The ROS-scavenging ability of BL and BL@B-SA₅₀ was measured in simulated gastric fluid (SGF), simulated intestinal fluid (SIF) and simulated inflammatory colon fluid (SICF) to assess whether the unfriendly conditions could decrease the catalytic activities. As shown in FIG. 5a-c, the ROS-scavenging ability of BL@B-SA₅₀ remained consistent in all of SGF, SIF and SICF for the indicated time points and were distinctly higher than that of BL, suggesting an extraordinary stability of BL@B-SA₅₀. The viability of BL was then measured in the above simulated gastrointestinal environment. Remarkably, arming with B-SA could improve the survival of BL in the inflammatory intestinal tract and would not compromise their tolerance in the acidic stomach and alkalic intestine (FIG. 5d-f). BL@B-SA not only enhances the resistance of BL to inflammation, but also maintains the ROS-scavenging ability, promising to alleviate the inflammatory environment and regulate gut microbiota.

It was further tested whether probiotics could enhance the targeting and retention of artificial enzymes in the intestine. Encouraged by the absorption of B-SA in the near-infrared region, its photoacoustic (PA) signal was investigated. Under a pulsed laser excitation at 808 nm, B-SA provided a strong PA contrast, which increased linearly as a function of concentration in the range of 25-100 μg/mL. Then, colitis mice were orally administered with equal amount of B-SA and BL@B-SA₅₀. As depicted in FIG. 5g, the PA signal of BL@B-SA₅₀ group at 808 nm was significantly higher than that of B-SA group, indicating more accumulation of BL@B-SA₅₀ in DSS-inflamed intestine at 6 h as a result of inherent intestinal colonization ability of BL. To further demonstrate the enhanced targeting ability of BL, the presence of nanomaterials was visualized using an in vivo imaging system (IVIS) at 6 hour post-administration. Due to the broad-spectrum absorption of B-SA in the NIR region, the fluorescence of DIR would be completely quenched. To address this issue, B-SA was replaced by the fluorescent DIR—B-Lip that consisted of targeting group of C18-B-PEG and fluorescent molecule of DIR. Then, DIR—B-Lip could be easily assembled on BL to form BL@DIR—B-Lip, with negligible fluorescence changes. Consistent with PA imaging results, orally administered BL@DIR—B-Lip could also accumulate in the DSS-inflamed colon more effectively than DIR—B-Lip, benefiting from the intestinal colonization abilities of BL. Taken together, the modification of BL could enhance the residence time of nanomaterials to achieve sustainable ROS-scavenging and inflammation-reducing performance.

### In Vivo Toxicity of BL@B-SA.

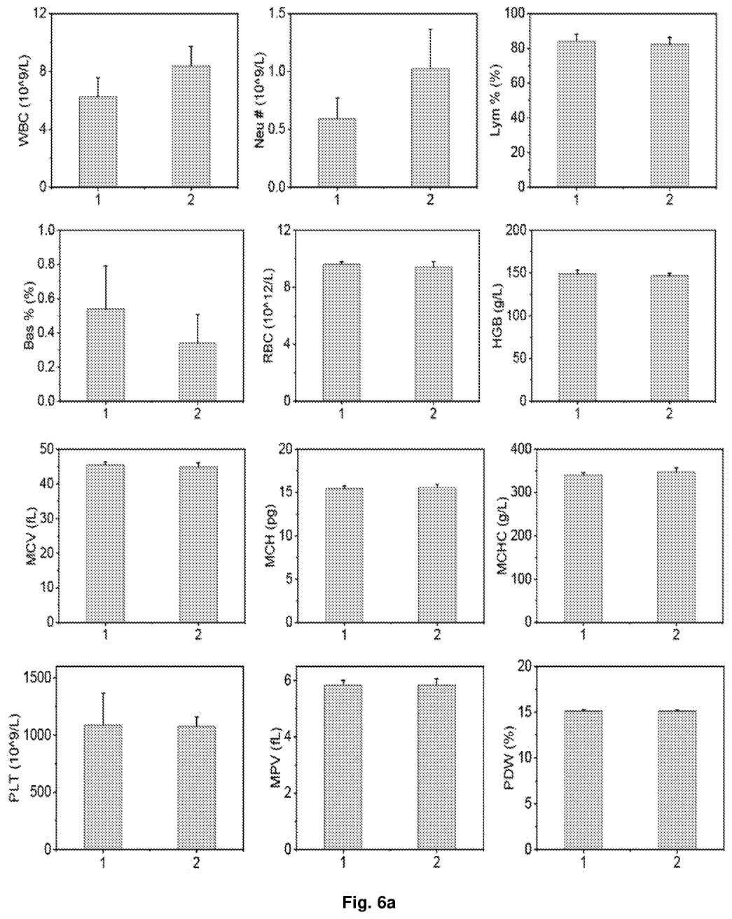

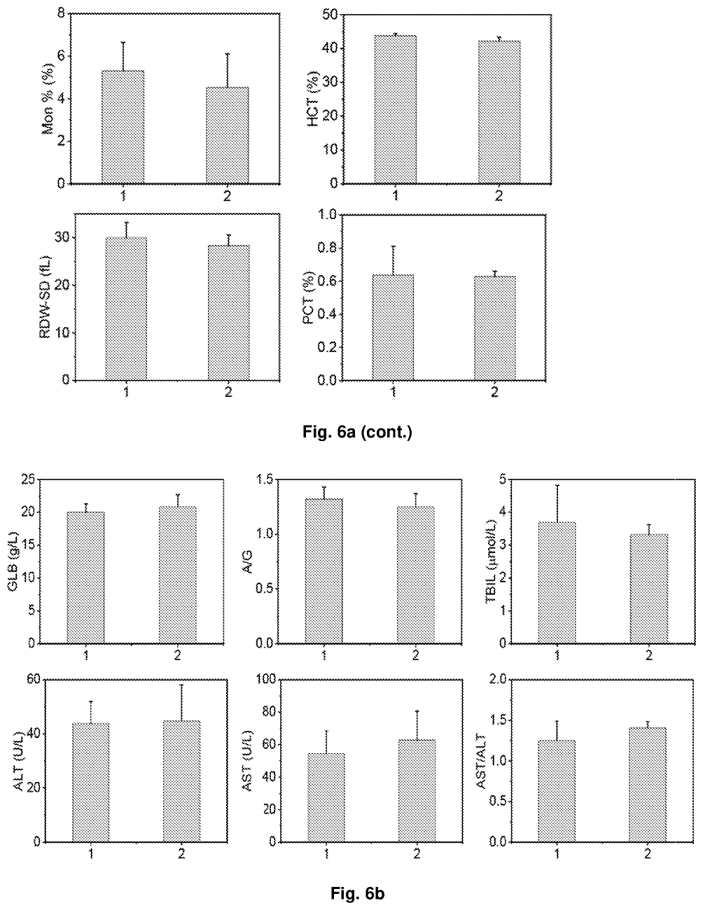

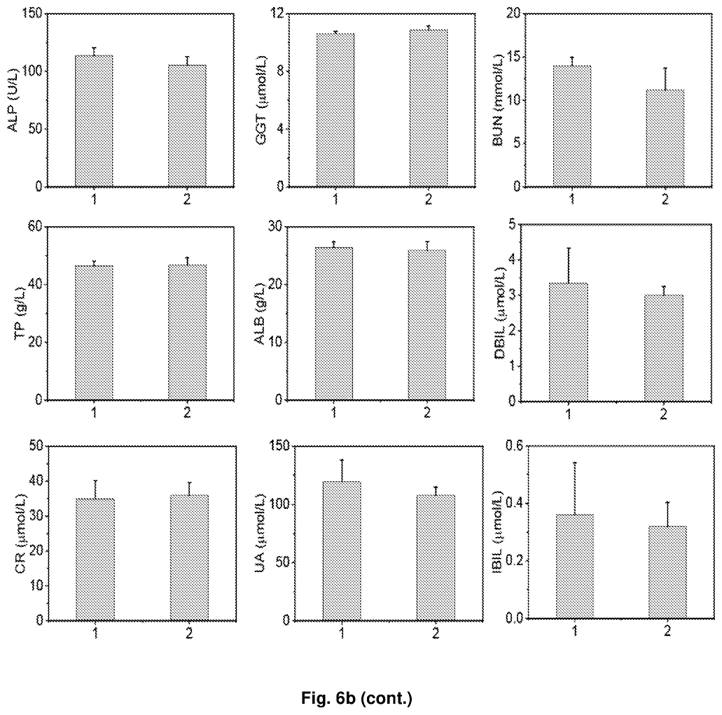

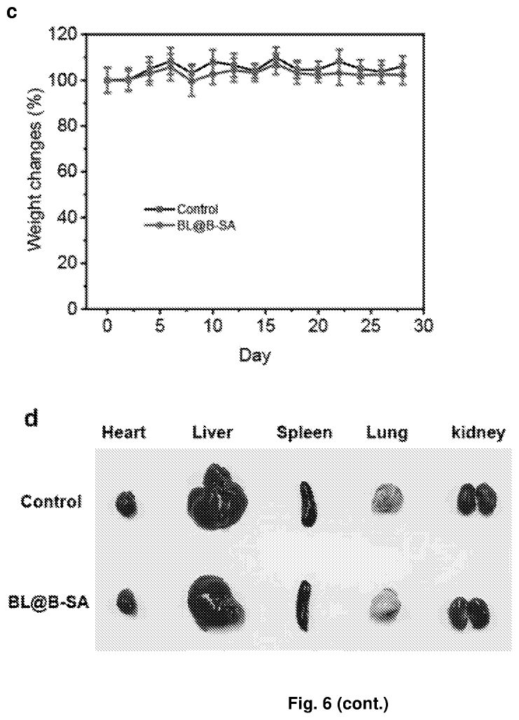

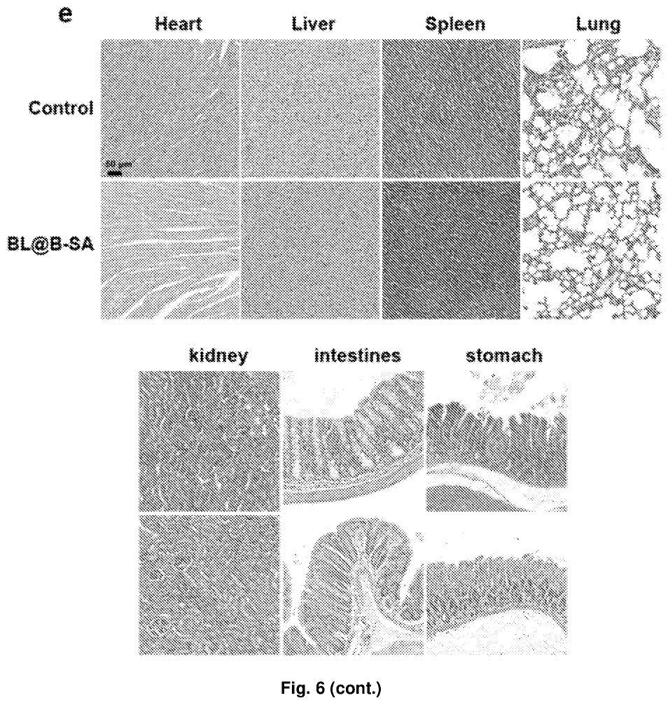

To ensure the safe bio-applications, in vivo toxicology of BL@B-SA₅₀ was systematically evaluated. The healthy mice were orally administered with or without BL@B-SA₅₀ daily for 28 days. Subsequently, their biochemical and hematological parameters, body weight changes as well as hematoxylin and eosin (H&E) staining of major organs were analyzed (FIG. 6a-c). There was no distinct difference between the BL@B-SA-treated group and the control group, suggesting negligible toxicity of BL@B-SA₅₀.

### Efficacy of BL@B-SA Against UC

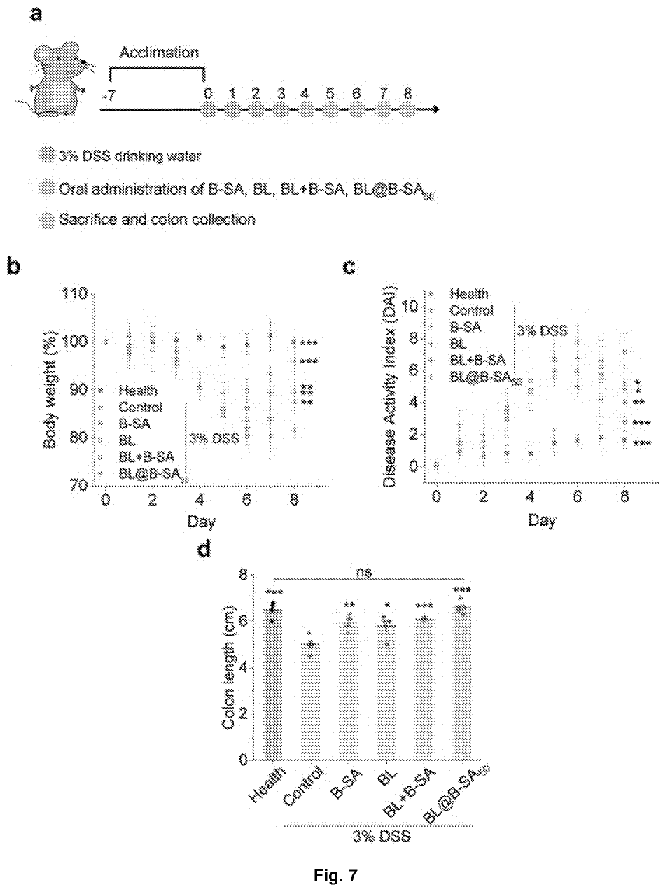

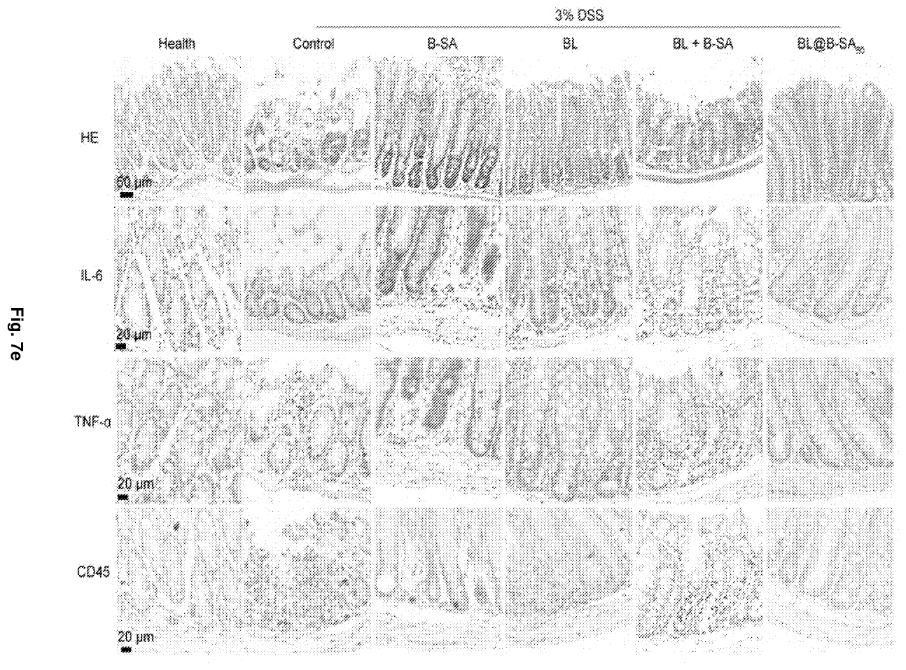

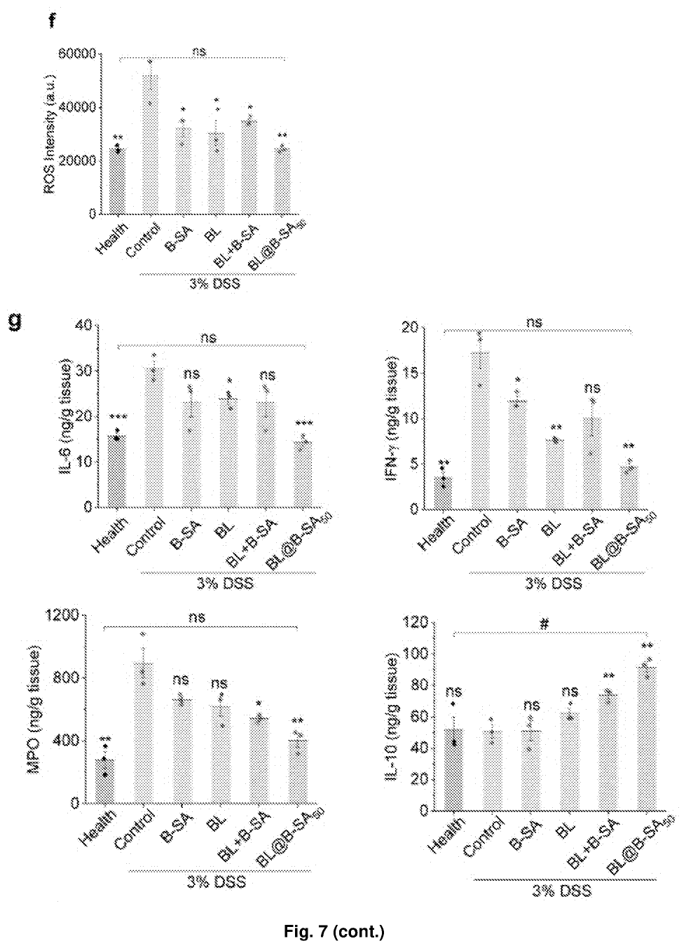

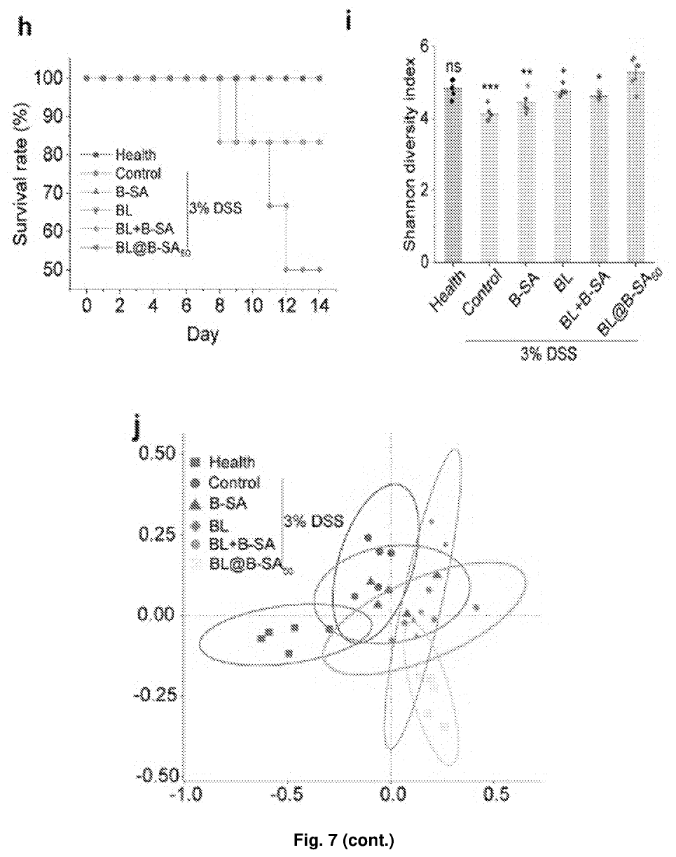

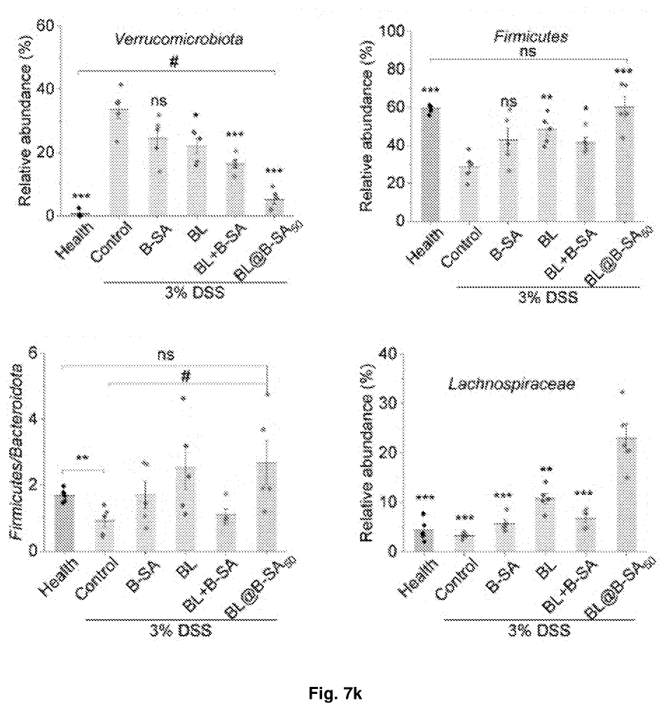

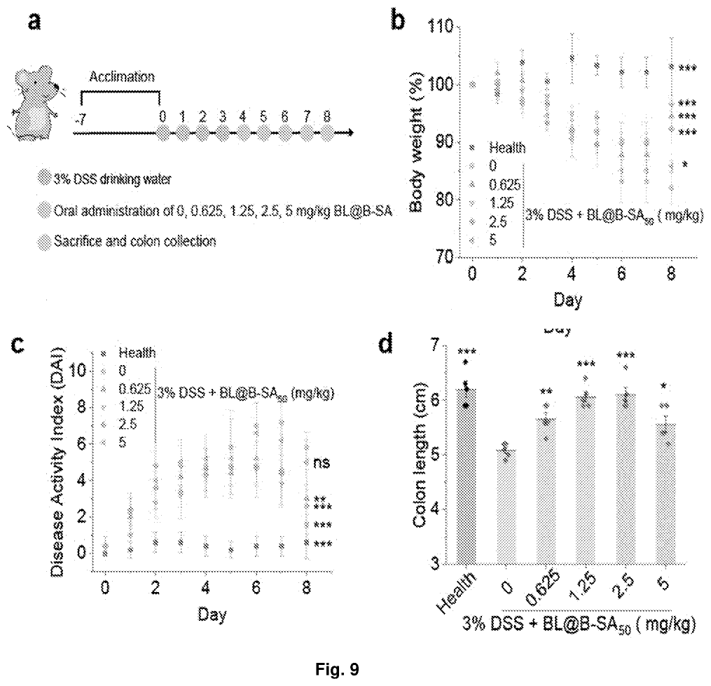

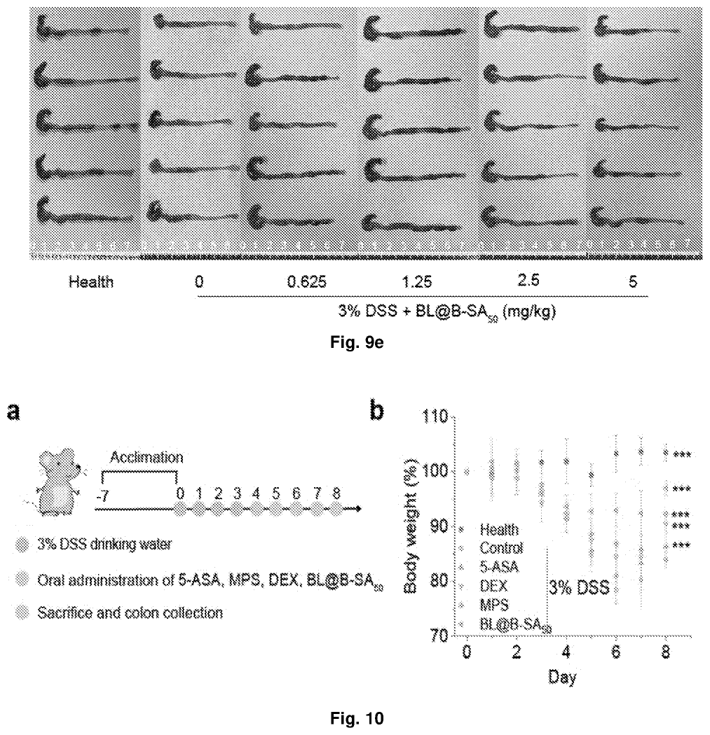

The therapeutic potential of BL@B-SA was then evaluated in vivo. Dextran sodium sulfate (DSS) is toxic to gut epithelial cells of the basal crypts and affects the integrity of the mucosal barrier. Mice were provided with 3% (w/v) DSS in drinking water for 4 consecutive days to induce colitis. To examine whether the proportions of BL and B-SA could influence the therapeutic outcomes of BL@B-SA, in the early stage of colitis, mice were orally administered with various BL@B-SA formulations every day for four days. BL@B-SA₅₀ (containing 2.5×10⁸ CFU/kg BL and 1.25 mg/kg B-SA) and BL@B-SA₁₀₀ (containing 2.5×10⁸ CFU/kg BL and 2.5 mg/kg B-SA) showed better protection for the animals against DSS-induced body weight loss, disease activity index increase, as well as shortening of colon length (FIG. 8). To achieve dose sparing with minimal side effects, the dosages of low formulated BL@B-SA₅₀ were also investigated (FIG. 9). Eventually, 1.25 mg/kg BL@B-SA₅₀ exerted a robust therapeutic effect against colitis and was chosen for further studies.

Whereafter, the efficacy of BL@B-SA₅₀ against other treatments was also evaluated (FIG. 7). After oral gavage, BL@B-SA₅₀ could predominantly interfere with the weight loss, DAI increase and colon length shortness associated with DSS-induced colitis, whereas other formulations showed reduced anti-IBD outcomes (FIG. 7b-d and 7l). Moreover, colitis mice treated with BL@B-SA₅₀ displayed normal feces, while colitis mice with other treatments developed symptoms including diarrhea and bloody stools (FIG. 7l). Importantly, the H&E staining and immunohistochemical analysis verified that BL@B-SA₅₀ treatment could protect colonic epithelium against pathological damage and suppress the expression of IL-6, TNF-α, and CD45 (FIG. 7e). Similarly, ROS experiments and ELISA analysis also confirmed that BL@B-SA₅₀ could reduce the levels of ROS and pro-inflammatory factors, including IL-6, IFN-γ and MPO, while increasing the levels of anti-inflammatory factors, such as IL-10 (FIGS. 7f and g). Finally, BL@B-SA₅₀ treatment could dramatically enhance the survival rate of UC mice (FIG. 7h) without triggering any overt signs of systemic toxicity, autoimmunity or pathologies in the major organs. Collectively, all these results demonstrated the superior efficacy of BL@B-SA₅₀ to BL, B-SA and BL+B-SA for colitis therapy due to the artificial enzymes-mediated potentiation of probiotic therapy.

### Modulation of Gut Microbiome

The modulation of the composition of gut microbiota in UC mice using BL@B-SA₅₀ was investigated as a form of therapy. Analyses of fecal samples by 16S ribosomal RNA (rRNA) gene sequencing in the V4 regions disclosed that BL@B-SA₅₀ treatment dramatically improved bacterial diversity in DSS-colitis mice, as indicated by the increase of the Shannon entropy index of a-diversity compared with the other groups (FIG. 7i). The non-metric multidimensional scaling (NMDS) plots revealed that UC mice treated with BL@B-SA₅₀ had distinct gut microbiota profile relative to the other treatment groups (FIG. 7j). Further analysis at the phylum/family level uncovered that BL@B-SA₅₀ treatment dramatically reduced the relative abundance of Verrucomicrobiota, increased relative abundance of Firmicutes and improved ratio of Firmicutes/Bacteroidota, reshaping a healthy gut microbiota (FIG. 7k). Interestingly, the relative abundance of Lachnospiraceae, that was included in Clostridium cluster XIVa and known to induce Treg cells, significantly increased after treatment with BL@B-SA₅₀ (FIG. 7k), thereby alleviating inflammation. Taken together, oral administration of SAzymes-armed probiotics BL@B-SA could increase the richness and diversity of gut microbiota to efficiently treat IBD.

Comparison with Clinical IBD Drugs

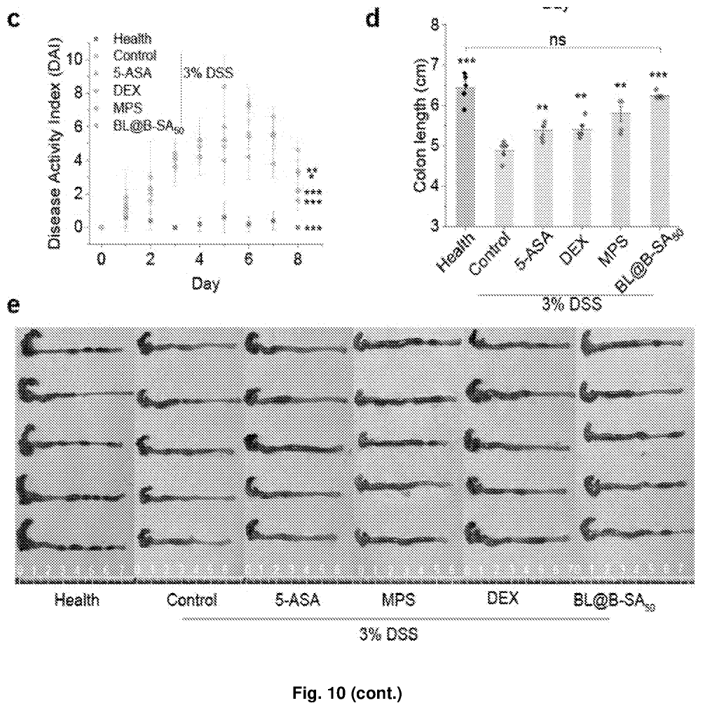

The efficacy of BL@B-SA was compared against other conventional IBD therapeutics widely used in the clinic, including 5-aminosalicylic acid (5-ASA), dexamethasone (DEX) and methylprednisolone (MPS). Outperforming 5-ASA, MPS and DEX used at their clinical doses, BL@B-SA exhibited significantly enhanced efficacy against DSS-induced acute colitis, achieving body weight recovery, reducing DAI and maintaining colon length (FIG. 10). BL@B-SA₅₀ is thus a promising candidate to replace clinical drugs for IBD treatment.

### Efficacy of BL@B-SA Against Chron's Disease (CD) on Mice

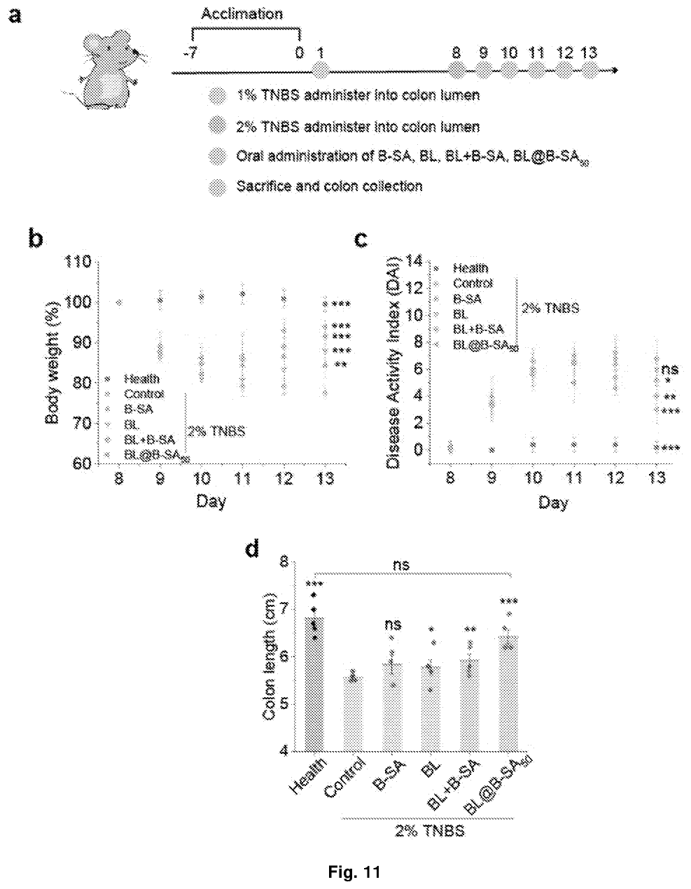

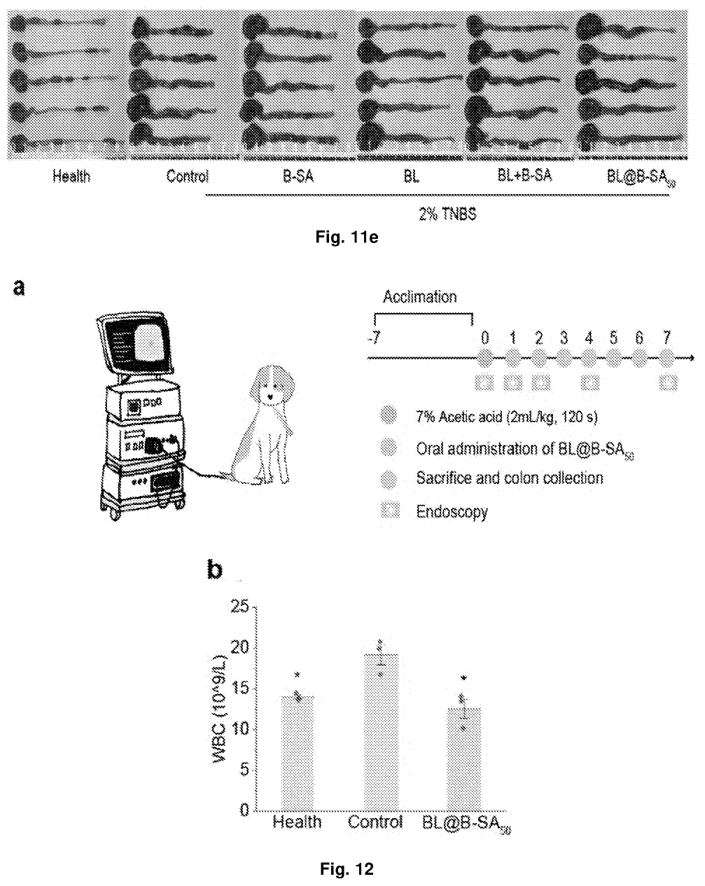

Chron's Disease (CD) is another major form of inflammatory bowel disease (IBD) in humans. To explore the scope of therapeutic applications of the probiotics-SAzyme, its efficacy was evaluated as a therapy for Chron's Disease (CD). Chron's Disease (CD) is induced by intrarectal administration of 2,4,6-trinitro benzene sulfonic acid (TNBS), which induces a T-cell-mediated response against hapten-modified autologous proteins/luminal antigens (FIG. 11a). Obvious body weight loss and bleeding were observed on day 9, suggesting the successful induction of Chron's Disease (CD) (FIG. 11b, c) in mice. Subsequently, Chron's Disease (CD) mice were orally administered with different materials for 4 days. Comparison of body weight changes, disease activity index (DAI) and colon lengths demonstrated the considerable therapeutic efficacy of BL@B-SA (FIG. 11b-c), which indicated greater efficacy of BL@B-SA over the other formulas.

### Efficacy Against Ulcerative Colitis (UC) on Dogs

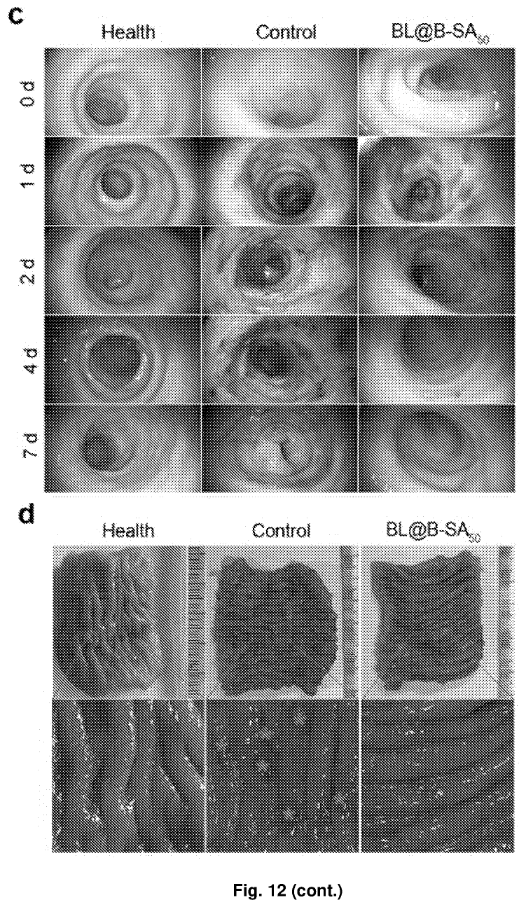

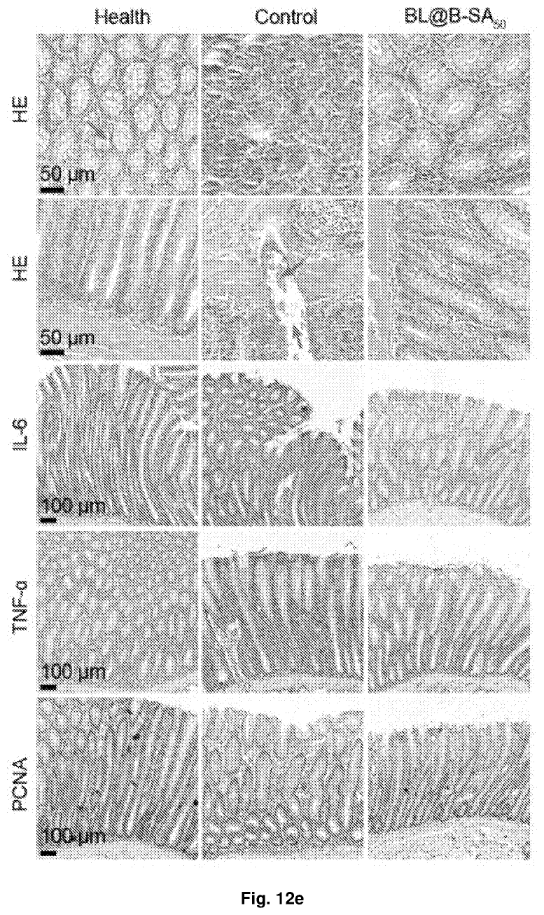

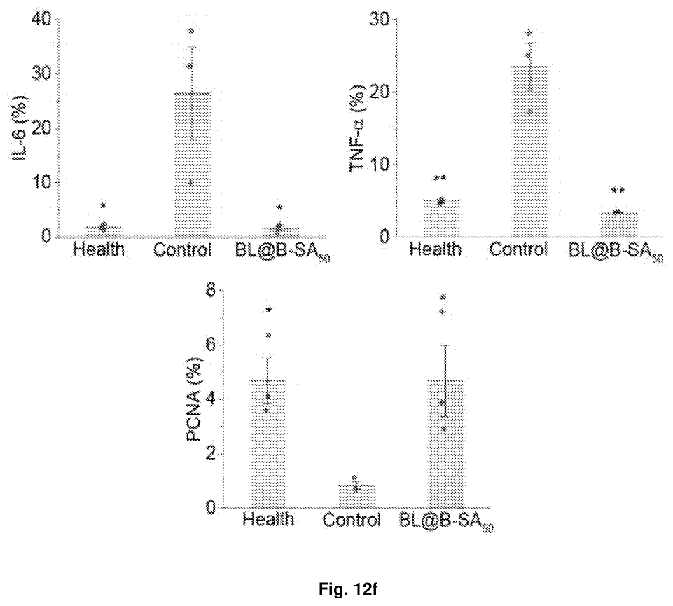

To promote the clinical translation of artificial enzymes-armed probiotics, the therapeutic potential was further evaluated in large animals with colitis. Canine UC model was built up by administering 7% acetic acid into the colon lumen of beagle dogs (FIG. 12a). Acetic acid could damage the intestinal tract and cause the production of inflammatory factors, which is similar to the transmission and type of inflammation in human colitis. After one day, compared with the smooth and pink colon in healthy dogs, obvious bleeding and cuts were observed in the colon of acetic acid-treated dogs by the endoscope, manifesting the successful establishment of the UC model (FIG. 12c). During therapy, oral administration of BL@B-SA₅₀ significantly promoted the healing of damaged colons, whereas the untreated UC dogs still showed wounds and bleeding (FIGS. 12c and d). Finally, compared to that of control groups, WBC levels in the BL@B-SA₅₀-treated group returned to normal after treatment, demonstrating the potential efficacy (FIG. 12b). Meanwhile, the H&E analysis also revealed the severe inflammation of untreated dogs, including inflammatory cell infiltration in the lamina propria, glandular destruction and goblet cell depletion (FIG. 12c). However, the inflammatory response was reduced after treatment with BL@B-SA₅₀, which was further confirmed by the immunohistochemical staining (FIG. 12e). The inflammation levels were further quantified by Image J, which was consistent with previous results (FIG. 12f). Overall, the results indicated that BL@B-SA was effective on the IBD of dogs, promising for their potential use in humans.

### Biosafety of BL@B-SA on Dogs.

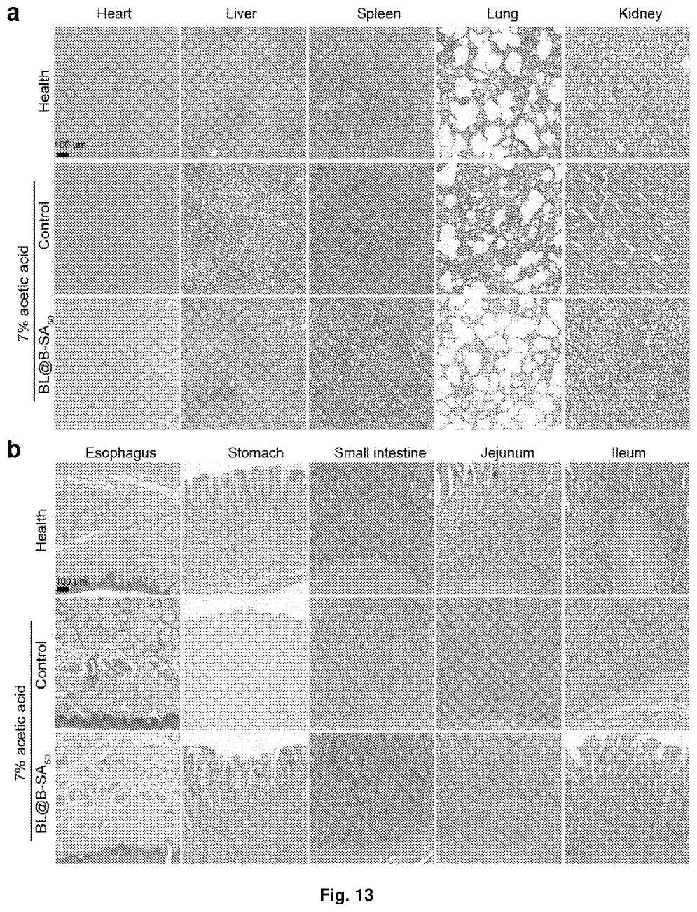

Finally, the biosafety of BL@B-SA was evaluated on dogs after treatment (FIG. 13). In the treatment, there were no significant body weight changes of dogs, indicating the safety of treatment ( ). Further, the H&E-stained major organs (heart, liver, spleen, lung and kidneys) and digestive tracts (esophagus, stomach, small intestine, jejunum and ileum) demonstrated a negligible difference between the healthy group and the BL@B-SA₅₀ treatment group (FIG. 13), indicating that formula would be suitable for large animals and humans.

### CONCLUSION

The rapid remission of inflammation and regaining the balance of gut microbiome are critical in treating gastrointestinal tract disorder such as, but not limited to inflammatory bowel disease (IBD) of ulcerative colitis, inflammatory bowel disease (IBD) of Crohn's disease or dysbiosis. However, conventional treatments such as the use of anti-inflammatory drugs and/or probiotics do not provide satisfactory outcomes. In addition, long term use of such anti-inflammatory drugs and/or probiotics can pose as a potential health hazard, which would decrease the patients' quality of life and increase the risk of more serious diseases, such as colon cancer.

The present application presents an engineered system comprising probiotic bacteria linker to artificial enzymes that have antioxidant abilities to rapidly and efficiently regulate the microenvironment of IBD (FIG. 2). In this system, artificial enzymes were employed to mimic the natural antioxidant defense systems to replace clinically used anti-inflammatory drugs. They scavenge ROS to reduce the inflammation (FIG. 4), with advantages of persistent cascade catalysis and biosafety over clinical drugs. In turn, the alleviated inflammatory environment improves the bacterium viability to rapidly reshape the intestinal barrier functions and gut microbiota (FIG. 4g and FIG. 7).

Furthermore, the retention time of nanomaterials is extended (FIG. 5g), which is due to the inherent colonization ability of probiotics. Thus, the system as disclosed herein, such as the BL@B-SA as described herein, provides the following benefits: (i) suppresses the levels of reactive oxygen species (ROS) and inflammation; (ii) restores the damaged intestinal barriers; and (iii) modulates the gut microbiome in local tissues. The therapeutic potential was demonstrated in DSS-induced ulcerative colitis (UC) (FIG. 7), which has outperformed other conventional therapies used in the clinic, such as 5-ASA, MPS and DEX (FIG. 10). Besides, the exemplary system BL@B-SA was also shown to alleviate the symptoms of TNBS-triggered Crohn's Disease (CD) (FIG. 11). Finally, the exemplary system BL@B-SA was also shown to be efficacious in acetic acid-linked UC in beagle dogs (FIG. 12), which is similar to the transmission and type of inflammation in human colitis. During the treatment, adverse events, such as opportunistic infections, autoimmunity and liver toxicity, were not observed in mice and dogs after repeated treatments using the exemplary system BL@B-SA (FIGS. 6 and 13). These results show that the system of the present application can be used as a therapeutic agent for treating different gastrointestinal tract disorders.

In summary, an artificial enzyme-probiotic bacteria system has been developed to regulate the microenvironment of gastrointestinal tract disorders such as inflammatory bowel disease (IBD). This system can effectively target inflamed colon, suppress the expression the reactive oxygen species (ROS) and inflammation, restore the damaged intestinal barriers and modulate the gut microbiome in local tissues. This work not only develops a new strategy to effectively treat gastrointestinal tract disorders such as inflammatory bowel disease (IBD), and also provides new anti-inflammatory probiotics to reduce inflammation and regulate the dysbiosis of microbiota.

1. A system comprising:
a) an artificial enzyme that facilitates a redox reaction;
b) a probiotic bacteria; and
c) a linker to conjugate the artificial enzyme and probiotic bacteria.

2. The system of claim 1, wherein the artificial enzyme comprises reactive oxygen species (ROS) scavenging ability to scavenge one or more reactive oxygen species (ROS).

3. The system of claim 2, wherein the artificial enzyme comprises reactive oxygen species (ROS) scavenging ability of reactive oxygen species (ROS) scavenging enzyme selected from a group consisting of superoxide dismutase (SOD), catalase (CAT) and glutathione peroxidase (GPx).

4. The system of claim 2, wherein the artificial enzyme comprises reactive oxygen species (ROS) scavenging ability of an antioxidant molecule selected from a group consisting of: glutathione (GSH), cysteine (Cys), polyphenol and vitamin C.

5. The system of any one of claims 1 to 4, wherein the artificial enzyme comprises a nanozyme.

6. The system of claim 5, wherein the nanozyme is selected from a group consisting of a single-atom nanozyme (SAzyme), a carbon-based nanozyme and a cluster-based nanozyme.

7. The system of any one of the preceding claims, wherein the linker comprises a phenylboronic acid functional group.

8. The system of claim 7, wherein the linker further comprises polyethylene glycol (PEG).

9. The system of any one of the preceding claims, wherein the probiotic bacteria is from a genus selected from a group consisting of Bifidobacterium, Lactobacillus, Komagataeibacter and Leuconostoc.

10. The system of claim 9, wherein the probiotic bacteria is Bifidobacterium.

11. The system of claim 10, wherein the Bifidobacterium is selected from a group consisting of Bifidobacterium longum, Bifidobacterium bifidum, Bifidobacterium breve, Bifidobacterium adolescentis, Bifidobacterium infantis, and Bifidobacterium animalis.

12. The system of any one of claims 1 to 11, wherein for every about 1×10⁷ CFU probiotic bacteria, the amount of artificial enzyme and linker comprises about 10 μg to about 500 μg.

13. The system of any one of claims 1 to 12, wherein for every about 1×10⁷ CFU probiotic bacteria, the amount of artificial enzyme and linker is about 50 μg.

14. A pharmaceutical composition comprising the system of any one of claims 1 to 13.

15. The pharmaceutical composition of claim 14, further comprising a pharmaceutically acceptable carrier.

16. A method of treating a disease in a subject in need thereof by administering a therapeutically effective amount of the system of any one of claims 1 to 13 or the pharmaceutical composition of claim 14 or 15.

17. The method of claim 16, wherein the disease is a gastrointestinal tract disorder.

18. The method of claim 17, wherein the gastrointestinal tract disorder is selected from a group consisting of inflammatory bowel disease (IBD) of ulcerative colitis, inflammatory bowel disease (IBD) of Crohn's disease, dysbiosis, necrotizing enterocolitis, acute infectious diarrhea, antibiotic-associated diarrhea, infant colic.

19. The method of any one of claims 16 to 18, comprising administering an effective amount of the system or the pharmaceutical composition comprising:
about 1.0×10⁷ to about 3.0×10⁸ CFU/kg probiotic bacteria; and
about 1.0 to about 3.0 mg/kg artificial enzyme and linker.

20. The method of any one of claims 16 to 19, comprising administering an effective amount of the system or the pharmaceutical composition comprising:
about 2.5×10⁸ CFU/kg probiotic bacteria; and
about 1.25 mg/kg artificial enzyme and linker.

21. A method of manufacturing the system as claimed in any one of claims 1 to 13, comprising incubating (i) an artificial enzyme conjugated to a linker; and (ii) a probiotic bacteria, wherein the linker conjugates the artificial enzyme and probiotic bacteria.
